(19) United States
(12) Patent Application Publication
(10) Pub. No.: US 2025/0279170 A1
(43) Pub. Date: Sep. 4, 2025

(54) AUTOMATED MEDICATION REVIEW RECONCILIATION

(71) Applicant: Kaiser Foundation Hospitals, Oakland, CA (US)

(72) Inventors: Loveena Harwani, Fremont, CA (US); Parveen Yadav, Oakland, CA (US); Ryan Wise, Colorado Springs, CO (US); Manabendra Das, Alpharetta, GA (US); Sarav Partha, Oakland, CA (US)

(21) Appl. No.: 19/067,529

(22) Filed: Feb. 28, 2025

Related U.S. Application Data
(60) Provisional application No. 63/559,525, filed on Feb. 29, 2024.
(60) Provisional application No. 63/635,873, filed on Apr. 18, 2024.

(51) Int. Cl. G16H 10/60; G16H 20/10; G16H 40/20; G16H 80/00

(52) U.S. Cl. CPC G16H 10/60; G16H 20/10; G16H 40/20; G16H 80/00

(57) ABSTRACT

A recommendation system for improved medical care powered by optimized care for medication reconciliation uses an artificial intelligence platform. Artificial Intelligence and machine learning (ML) based approaches significantly optimize the care delivered to a patient user and efficiently utilizes medication reconciliation workflows in healthcare organizations. By identifying a patient, enabling automatic reminders to complete medication reviews before, during, and after visits, care delivery with respect to medication reconciliation is optimized.

### CROSS-REFERENCE TO RELATED APPLICATION

This application claims priority to U.S. Provisional Patent Application Ser. No. 63/559,525, filed on Feb. 29, 2024 and to U.S. Provisional Patent Application Ser. No. 63/635,873, filed on Apr. 18, 2024, which are both hereby incorporated by reference in their entirety.

### BACKGROUND

This specification generally relates to a system and method for providing improved medical care. In particular, the specification relates to a system and method for providing improved medical care by automatically reconciling medication review.

Healthcare organizations, healthcare providers, physician offices, and hospitals continuously receive healthcare-related requests from users. Additionally, many users are beginning to interact with these entities via new technologies or channels such as via the web, video, online messaging, email, texting, in addition to the more traditional ways of interacting such as telephone or in-person conversations or appointments. Several issues arise with so many new and different ways of patient-entity interactions. The interactions are siloed without channel cross-over, limited options and lack of understanding, missed opportunities with human interactions, and an inability to seamlessly transition to another channel. Without channel integration and guidance, the users may have to exit and choose a new channel. Furthermore, the incoming messages are neither organized nor prioritized; and therefore, require a significant amount of human and other processing to identify the patient's needs and respond in a timely manner.

One issue is with all these new ways of interacting with healthcare providers is ensuring that users or patients understand and can perform all the routine visits, screening, and other actions necessary to keep them in good health after a visit.

Another issue is ensuring that the past visits to any healthcare facility are effectively organized to make sure that the patient can complete as many tasks as necessary before and after in-facility, telephonic, or video visits.

One final issue is making sure that users and patients are effectively reminded of new health tasks, including reviewing prescriptions for medicine that are currently being taken, that need to be completed before and/or after a facility visit. Upon their next facility visit, a medication review is often a cumbersome process where the patient member may forget the name of a medication or forget whether a new medication had been suggested previously.

This background description provided herein is for the purpose of generally presenting the context of the disclosure.

### SUMMARY

The techniques introduced herein overcome the deficiencies and limitations of the prior art, at least in part, with a system and methods providing improved medical care by automating medication review. In one implementation, the system and method of the present disclosure identify the user/patient, with their consent, and based upon an analysis of their medical care needs using artificial intelligence (AI)/machine learning (ML) classification models, identifies care gaps and service availability and provides recommendations for additional services. The system and method of the present disclosure may advantageously improve member/patient experience in real-time.

In some implementations, the systems and methods of the present disclosure generate and send automatic reminders about medical tasks that may be performed before their next visit, either in person at a medical facility or remotely via phone or video call. The systems and methods advantageously encourage patients to review medications through a conversational interface application, reducing the burden for care providers to take time during a visit to manually review medications being taken by patients. In particular, in some implementations, the systems and methods remind patients about completing medication review tasks that can be performed before an upcoming visit or after an existing visit. For example, a reminder about a medication review task that has not yet been performed can be generated as a notification and sent to a client device associated with the patient. Then, the patient can be prompted to agree to perform that task before a visit, and the systems and methods automatically perform all underlying requirements of the medication review task, including retrieving medication data, performing analysis based on user input, providing the medication data to a care provider, etc. The systems and methods can automatically determine unmet care needs for the patient that can be performed before or after a visit. The systems and methods generate and send notifications to the client device of the patient and interact with the client device to complete the action after a visit.

The features and advantages described herein are not all-inclusive and many additional features and advantages will be apparent in view of the figures and description. Moreover, it should be understood that the language used in the present disclosure has been principally selected for readability and instructional purposes, and not to limit the scope of the subject matter disclosed herein.

### BRIEF DESCRIPTION OF THE DRAWINGS

The disclosure is illustrated by way of example, and not by way of limitation in the figures of the accompanying drawings in which like reference numerals are used to refer to similar elements.

FIG. 1 is a high-level block diagram illustrating one example implementation of a system for post-visit action optimization.

FIG. 2 is a block diagram illustrating one implementation of a computing device including a post-visit action module.

FIG. 3A is a high-level block diagram showing one implementation for the conversational care optimization module.

FIG. 3B is a high-level block diagram showing one implementation for the automatic medication reconciliation module.

FIG. 4 is a block diagram illustrating an example implementation of the data storage and data sources.

FIG. 5 is a block diagram illustrating one implementation of a care optimization engine.

FIG. 6 is a flowchart illustrating one implementation of a method for automating medication reconciliation.

FIG. 7 is a flowchart illustrating one implementation of a method for notifying the patient to automate medication reconciliation.

FIG. 8 is a flowchart illustrating one implementation of a method for determining post-visit analysis and prompting a patient with a suggested action.

FIG. 9 is a flowchart illustrating one implementation of a method for determining medication data analysis and confirming with a patient post-visit.

FIGS. 10A-10B are example implementations of graphical user interfaces that may be displayed in accordance with the present invention.

FIGS. 11A-11C are example implementations of graphical user interfaces that may be displayed in accordance with the present invention.

### DETAILED DESCRIPTION

While the present disclosure may describe the techniques herein in the context of a patient member seeking healthcare services in hospitals, medical clinics and the like, it should be understood that the architecture, principles, and components of the present disclosure may also be used to provide care optimization, service gaps, and customer service in a variety of other contexts. For example, the present disclosure may be applied to retail sales, technical support services, insurance, a hospitality organization, any other areas where location, gaps in service or care and notifications are important. The systems and methods described below may be applied to various other medical care, coordination, and delivery procedures in addition to those specifically set forth below.

In the realm of healthcare, the management of patient medication is a complex and multifaceted task. Primary care physicians and other healthcare providers are tasked with gathering and understanding a vast amount of information about a patient's medication regimen, including the types of medications being taken, the dosage and frequency of each medication, and the patient's adherence to this regimen. This process is time-consuming and can be prone to errors, particularly when a patient is taking multiple medications.

Furthermore, patients often face challenges in adhering to their medication regimen. These challenges can include side effects, difficulties in remembering to take the medication, and confusion about the timing and dosage of each medication. These issues can lead to suboptimal treatment outcomes and can negatively impact the patient's health.

Communication between healthcare providers and patients about medication is also a challenge. Traditional methods of communication, such as in-person appointments and phone calls, can be time-consuming and inconvenient for both parties. Furthermore, these methods do not allow for real-time monitoring of a patient's medication adherence, which can be valuable in identifying and addressing issues early on.

There is a clear and pressing demand for a solution that can streamline the process of medication management, improve communication between healthcare providers and patients, and enhance patient adherence to their medication regimen. Such a solution would ideally be able to handle multiple medications for a single patient, identify potential issues or barriers to medication adherence, and suggest interventions to address these issues. It would also be able to switch between different channels of communication, such as text or voice, depending on the patient's preference.

However, despite the clear demand for such a solution, the development and implementation of such a system presents numerous challenges. These challenges include the complexity of handling multiple medications for a single patient, the difficulty of accurately assessing medication adherence, and the technical challenges associated with developing a system that can understand and respond to user input in multiple languages and through multiple channels of communication.

The disclosed method involves a conversational interface application that interacts with a patient member user in a healthcare system. The application receives user input, which can be in various forms such as text or voice commands, and uses this input to determine the patient's health conditions and current medication usage. The application then presents questions related to these health conditions and medications, and receives responses from the patient. Based on these responses, the application determines recommended actions for the patient to manage their health conditions and optimize their treatment outcomes.

The user input can include information about the patient's adherence to their prescribed medication regimen, such as the timing, dosage, and frequency of medication intake. The application uses this information to assess the patient's adherence and to inform the recommended actions. These actions can include a variety of different types of actions, such as changes to the patient's medication regimen, scheduling of follow-up appointments, or referrals to other healthcare services.

The application is capable of handling multiple medications for a single patient and can provide appropriate recommendations and actions based on the complex interplay of these multiple medications. The application can also identify potential issues or barriers to medication adherence, such as side effects or difficulties with remembering to take the medication and can suggest interventions to address these issues.

The application is designed to be used on various devices such as smartphones, tablets, computers, and can receive and understand user input in multiple languages. It also has the capability to switch between different channels of communication, such as text or voice, depending on the patient's preference.

User Input may be defined as any information or data provided by the patient member user through the conversational interface application. This could include, but is not limited to, text, voice commands, selections from multiple-choice options, or any other form of data entry supported by the application. Herein, a conversational interface application may be a software application that allows users to interact with it using natural language. This interaction can be in the form of text or voice, and the application is designed to understand and respond to user input in a conversational manner. The application can be accessed through various devices such as smartphones, tablets, computers, or any other device capable of running software applications.

As used herein, a patient member user is an individual who is using the conversational interface application and is a member of a healthcare system or service. This individual is typically a patient who is seeking or receiving healthcare services and is using the application to manage their health conditions, medications, appointments, or other aspects of their healthcare.

The term current medication usage may refer to information about the medications that the patient member user is currently taking or has been prescribed. This can include details such as the names of the medications, the dosages, the frequency of intake, the method of administration, the duration of the treatment, and any other relevant information. This information is typically provided by the patient member user as part of their user input to the conversational interface application. Chronic diseases are long-term health conditions that persist for a period of time, typically longer than three months. Chronic diseases may require ongoing medical attention or limit an individual's activities. Examples of chronic diseases include, but are not limited to, heart disease, diabetes, chronic respiratory diseases, and cancer. Long-term medication refers to drugs or other pharmaceutical compounds that are prescribed to be taken regularly over a prolonged period of time, often for the management of chronic diseases. The duration of this medication regimen can vary based on the specific health condition, the patient's response to the medication, and the healthcare provider's recommendations.

Health condition determination is the process of identifying or diagnosing a patient's health condition or disease. This determination is made based on the user input received through the conversational interface application. This could involve analyzing the user's reported symptoms, medical history, or other relevant information. Frequency of medication intake refers to how often a patient member user is instructed to take a prescribed medication. This could be several times a day, once a day, weekly, or any other interval as prescribed by a healthcare provider. Dosage of medication refers to the amount of a particular medication that a patient member user is instructed to take at each instance. Dosage is typically measured in milligrams (mg), but can also be in other units such as units, milliliters (ml), or number of pills, among others. Timing of medication intake refers to the specific times at which a patient member user is instructed to take a prescribed medication. This could be at specific times of the day (e.g., morning, afternoon, evening), in relation to meals (e.g., before meals, after meals), or at other specific intervals (e.g., every 12 hours, every 24 hours).

Adherence to the prescribed medication regimen refers to the extent to which a patient member user follows the instructions or recommendations provided by their healthcare provider regarding the use of their prescribed medication. This includes factors such as the timing, dosage, and frequency of medication intake, as well as any specific instructions related to the administration of the medication (e.g., taking the medication with or without food, avoiding specific activities or substances while on the medication, etc.). Adherence can be influenced by a variety of factors, including the patient's understanding of their medication regimen, their ability to remember to take their medication, their motivation to follow the regimen, and any barriers or challenges they may face in doing so.

A conversational interface application is a software application that allows users to interact with it using natural language. This interaction can be in the form of text or voice, and the application is designed to understand and respond to user input in a conversational manner. The application can be accessed through various devices such as smartphones, tablets, computers, or any other device capable of running software applications. In the context of this patent, the conversational interface application is used to gather information about the patient member user's adherence to their prescribed medication regimen.

Information provided by the patient member user is made in one or more responses to the questions presented through the conversational interface application. These responses can include direct answers to the questions, as well as any additional information or comments that the patient member user chooses to provide. The responses are used to assess the patient's adherence to their prescribed medication regimen and to inform the recommended actions for managing their health conditions. Recommended actions are suggestions or advice provided to the patient member user based on the analysis of their responses to the questions presented through the conversational interface application. These actions are intended to help the patient manage their health conditions and optimize their treatment outcomes. They can include a variety of different types of actions, such as changes to the patient's medication regimen, scheduling of follow-up appointments, or referrals to other healthcare services.

Medication adjustments are changes or modifications to a patient's prescribed medication regimen. This could include changes to the dosage of the medication, the frequency of intake, the timing of intake, or the method of administration. Medication adjustments are typically recommended by a healthcare provider based on the patient's responses to the questions presented through the conversational interface application, as well as other relevant information such as the patient's health status, the effectiveness of the current medication regimen, and any side effects or adverse reactions the patient may be experiencing.

Access to health care refers to the ability of a patient to obtain medical services. In the context of this patent, access to health care is facilitated by the recommended actions presented to the patient member user through the conversational interface application. These actions are designed to help the patient manage their health conditions and optimize their treatment outcomes and can include a variety of different types of actions such as scheduling appointments, providing medication information, or facilitating communication with healthcare providers.

Follow-up appointments are scheduled meetings with a healthcare provider that occurs after an initial consultation or previous appointment. A follow-up appointment is recommended to the patient member user through the conversational interface application, specifically for discussing the at least one medication managing the one or more health conditions. A professional who provides preventive, curative, promotional, or rehabilitative health care services in a systematic way to people, families, or communities is a healthcare provider. The healthcare provider is the individual who prescribes at least one medication to the patient member user and with whom the patient member user has a follow-up appointment to discuss the medication.

A conversation or dialogue occurs between the patient member user and the healthcare provider about at least one medication. This discussion can include, but is not limited to, topics such as the effectiveness of the medication, any side effects or adverse reactions, the patient's adherence to the medication regimen, and any concerns or questions the patient may have about the medication. The medication discussion is recommended to occur during a follow-up appointment with the healthcare provider, in an embodiment.

A machine learning-based scoring pipeline is a sequence of data processing steps or stages that utilizes machine learning algorithms to assign scores or values to data. Here, the scoring pipeline is used to perform text classification and entity recognition on the user input received through the conversational interface application. Text classification is a process of categorizing text into predefined groups or categories based on its content. Text classification is used to analyze the user input received through the conversational interface application and to determine the relevant health conditions and medication usage. Entity recognition, also known as Named Entity Recognition (NER), is a subtask of information extraction that seeks to locate and classify named entities in text into predefined categories such as person names, organizations, locations, medical codes, time expressions, quantities, monetary values, percentages, etc. Entity recognition is used to identify specific details or elements in the user input, such as the names of medications, the dosages, the frequency of intake, and other relevant information.

A software application designed to run on mobile devices such as smartphones and tablets, a mobile application is the conversational interface application that is used to gather and present information about the patient member user's medication regimen and health conditions. Multi-lingual user input is user input that is provided in multiple languages. The conversational interface application can receive and interpret user input in multiple languages, allowing it to be used by patient member users who speak different languages, in an embodiment.

Channel switching is the ability of a software application to switch between different modes or channels of communication based on user preference. The conversational interface application can switch between different channels such as text, voice, or other forms of communication, depending on the patient member user's preference.

“One or more health conditions” refers to any medical condition, disease, disorder, or ailment that a patient may have. These health conditions are managed by at least one medication, which means that the patient is prescribed or is taking one or more drugs or medicines to treat, manage, or control these health conditions. The medication could be in various forms such as tablets, capsules, liquids, injections, or topical applications, among others.

The management of health conditions by medication involves a range of activities. These can include the initial prescription of the medication by a healthcare provider, the dispensing of the medication by a pharmacist, the administration or consumption of the medication by the patient, and the ongoing monitoring of the patient's response to the medication. This monitoring can involve assessing the effectiveness of the medication in treating or managing the health condition, as well as monitoring for any side effects or adverse reactions to the medication.

In some cases, a health condition may be managed by more than one medication. This could be because the condition requires a combination of drugs for effective treatment, or because the patient has multiple health conditions that each require different medications. In such cases, the disclosed method is capable of handling multiple medications for a single patient and can provide appropriate recommendations and actions based on the complex interplay of these multiple medications.

The one or more responses received through the conversational interface application include information about the patient member user's adherence to the prescribed medication regimen. This information can be gathered in various ways, such as through direct questions about the patient's medication usage, or through indirect indicators such as the patient's reported symptoms or health status. The responses can provide valuable insights into whether the patient is taking the medication as prescribed, whether they are experiencing any side effects or difficulties with the medication, and whether they have any concerns or questions about the medication. This information can then be used to inform the healthcare provider's decisions about the patient's treatment plan, including any adjustments to the medication regimen that may be warranted.

Furthermore, the responses can also be used to identify any potential issues or barriers to medication adherence. For example, if a patient reports that they are not taking their medication as prescribed because they are experiencing side effects, this could indicate a potential issue with the medication that may require further investigation. Similarly, if a patient reports that they are not taking their medication as prescribed because they are having difficulty remembering to take it, this could indicate a potential barrier to adherence that could be addressed through interventions such as medication reminders or pill organizers.

Overall, the responses received through the conversational interface application can provide a comprehensive picture of the patient's medication adherence, which can be invaluable in managing their health conditions and optimizing their treatment outcomes.

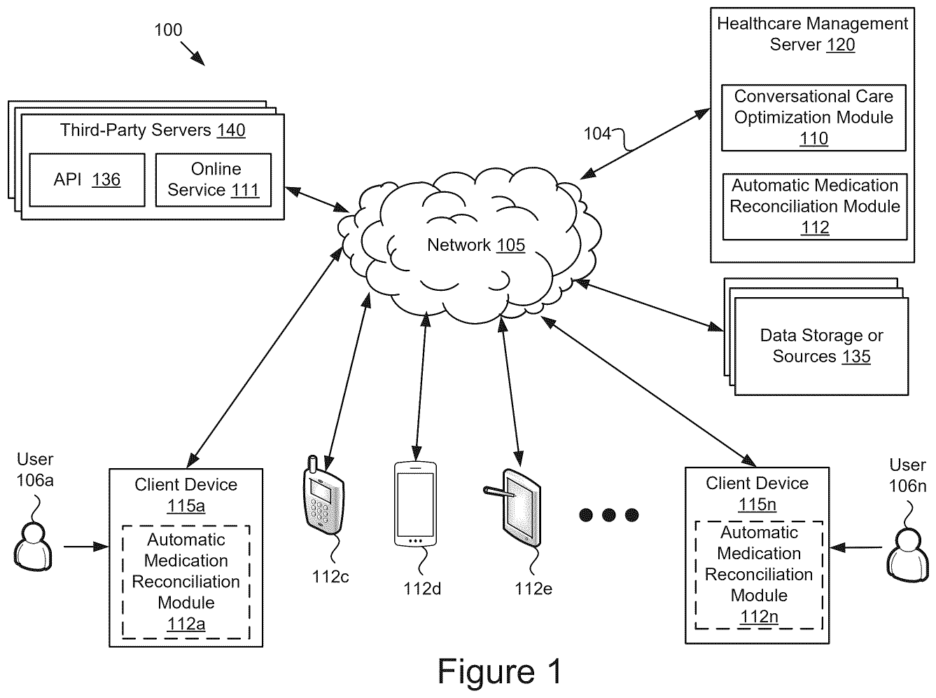

FIG. 1 is a high-level block diagram illustrating one implementation of a system 100 for providing improved medical care by automating medication reconciliation. The illustrated system 100 may include one or more client devices 115a . . . 115n that can be accessed by users, a healthcare management server 120, a plurality of data sources 135, and a plurality of third-party servers 140 which are communicatively coupled via a network 105 for interaction and electronic communication with one another. In FIG. 1 and the remaining figures, a letter after a reference number, e.g., “115a,” represents a reference to the element having that particular reference number. A reference number in the text without a following letter, e.g., “115,” represents a general reference to instances of the element bearing that reference number.

The network 105 may be a conventional type, wired or wireless, and may have numerous different configurations including a star configuration, token ring configuration, or other configurations. Furthermore, the network 105 may include any number of networks and/or network types. For example, the network 105 may include a local area network (LAN), a wide area network (WAN) (e.g., the Internet), virtual private networks (VPNs), mobile (cellular) networks, wireless wide area network (WWANs), WiMAX® networks, Bluetooth® communication networks, peer-to-peer networks, near field networks (e.g., NFC, etc.), and/or other interconnected data paths across which multiple devices may communicate, various combinations thereof, etc. The network 105 may also be coupled to or include portions of a telecommunications network for sending data in a variety of different communication protocols. In some implementations, the network 105 may include Bluetooth communication networks or a cellular communications network for sending and receiving data including via short messaging service (SMS), multimedia messaging service (MMS), hypertext transfer protocol (HTTP), direct data connection, WAP, email, etc. In some implementations, the data transmitted by the network 105 may include packetized data (e.g., Internet Protocol (IP) data packets) that is routed to designated computing devices coupled to the network 105. Although FIG. 1 illustrates one network 105 coupled to the client devices 115, the healthcare management server 120, the plurality of data sources 135, and the plurality of third-party servers 140, in practice one or more networks 105 can be connected to these entities.

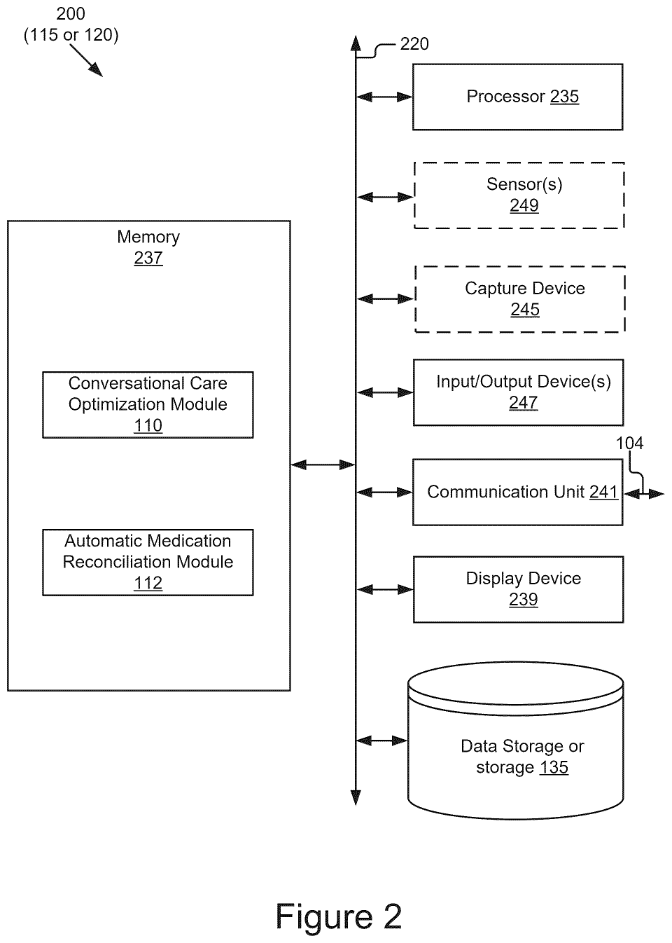

The client devices 115a . . . 115n (also referred to individually and collectively as 115) may be computing devices having data processing and communication capabilities. In some implementations, a client device 115 may include a memory, a processor (e.g., virtual, physical, etc.), a power source, a network interface, software and/or hardware components, such as a display, graphics processing unit (GPU), wireless transceivers, keyboard, camera (e.g., webcam), sensors, firmware, operating systems, web browsers, applications, drivers, and various physical connection interfaces (e.g., USB, HDMI, etc.). The client devices 115a . . . 115n may couple to and communicate with one another and the other entities of the system 100 via the network 105 using a wireless and/or wired connection. Examples of client devices 115 may include, but are not limited to, laptops, desktops, tablets, mobile phones (e.g., smartphones, feature phones, etc.), server appliances, servers, virtual machines, smart TVs, media streaming devices, user wearable computing devices (e.g., fitness trackers, etc.) or any other electronic device capable of accessing a network 105. In the example of FIG. 1, the client device 115a is configured to implement a conversational care optimization module 110 described in more detail below. The client device 115 includes a display for viewing information provided by one or more entities coupled to the network 105. For example, the client device 115 may be adapted to send and receive data to and from the healthcare management server 120. While two or more client devices 115 are depicted in FIG. 1, the system 100 may include any number of client devices 115. In addition, the client devices 115a . . . 115n may be the same or different types of computing devices. The client devices 115a . . . 115n may be associated with the users 106a . . . 106n. For example, users 106a . . . 106n may include patient members, physicians, clinical staff, laboratory technicians, pharmacy technicians, administrative staff, call center agents, etc. of a health care organization. Each client device 115 may be associated with a data channel, such as a mobile application running on a user's smartphone, a computer in a doctor's office, a health tracking device, etc. These data channels may collect data related to one or more users and provide that data to the entities coupled to the network 105. In some implementations, the client devices 115 may be implemented as a computing device 200 as will be described below with reference to FIG. 2.

In the example of FIG. 1, the healthcare management server 120, the plurality of data sources 135, and the plurality of the third-party servers 140 may be, or may be implemented by, a computing device including a processor, a memory, applications, a database, and network communication capabilities similar to that described below with reference to FIG. 2.

In the example of FIG. 1, the healthcare management server 120 may be configured to implement a conversational care optimization module 110 and an automatic medication reconciliation module 112. In some implementations, the healthcare management server 120 may be a hardware server, a software server, or a combination of software and hardware. For example, the healthcare management server 120 may include one or more hardware servers, virtual servers, server arrays, storage devices and/or systems, etc., and/or may be centralized or distributed/cloud-based. In some implementations, the healthcare management server 120 may include one or more virtual servers, which operate in a host server environment and access the physical hardware of the host server including, for example, a processor, a memory, applications, a database, storage, network interfaces, etc., via an abstraction layer (e.g., a virtual machine manager). In some implementations, the healthcare management server 120 may be a Hypertext Transfer Protocol (HTTP) server, a Representational State Transfer (REST) service, or other server type, having structure and/or functionality for processing and satisfying content requests and/or receiving content from one or more of the client devices 115, the plurality of data sources 135, and the plurality of third-party servers 140 that are coupled to the network 105.

Also, instead of or in addition, the healthcare management server 120 may implement its own application programming interface (API) for the transmission of instructions, data, results, and other information between the server 120 and other entities communicatively coupled to the network 105. For example, the API may be a software interface exposed over the HTTP protocol by the healthcare management server 120. The API exposes internal data and functionality of the service hosted by the healthcare management server 120 to API requests originating from one or more of the conversational care optimization module s 110, the plurality of data sources 135, and the plurality of third-party servers 140. In one example, the conversational care optimization module 110 implemented by the healthcare management server 120 receives messages from the client devices 115 and sends information and action requests in response. In some implementations, the conversational care optimization module 110 passes an authenticated request including a set of parameters for information to one or more of the third-party servers 140 and the data source 135 and receives an object (e.g., XML or JSON) with associated results. In some implementations, the healthcare management server 120 may also include a database coupled to it (e.g., over the network 105) to store structured data in a relational database and a file system (e.g., HDFS, NFS, etc.) for unstructured or semi-structured data. In some implementations, the healthcare management server 120 may include an instance of a data store that stores various types of data for access and/or retrieval by the conversational care optimization module 110. For example, the data store may store machine learning models for natural language understanding of user intents, actions associated with messages, and other information as will be described below. Other types of user data are also possible and contemplated.

In some implementations, the healthcare management server 120 sends and receives messages and data to and from other entities of the system 100 via the network 105. For example, the healthcare management server 120 sends and receives messages including instructions to and from the client device 115. In some implementations, the healthcare management server 120 may serve as a middle layer and permit interactions between the client device 115 and the plurality of the third-party servers 140 and the data sources 135 to flow through and from the healthcare management server 120 for security and convenience. In some implementations, the healthcare management server 120 may be operable to receive a message or series of messages from a client device 115, determine user intent from those messages, and initiate an action that is responsive to the message or messages. For example, the message intent may be for appointment request, the healthcare management server 120 processes the appointment request based on the user context and hospital resource availability, and generates and sends a reply message with a recommended appointment for treating a patient health condition determined from the message or messages, etc. The healthcare management server 120 may send data to and receive data from the other entities of the system 100 via the network 105. It should be understood that the healthcare management server 120 is not limited to providing the above-noted acts and/or functionality and may include other network-accessible services. In addition, while a single healthcare management server 120 is depicted in FIG. 1, it should be understood that there may be any number of healthcare management servers 120 or a server cluster.

Each of the one or more third-party servers 140 may be, or may be implemented by, a computing device including a processor, a memory, applications, a database, and network communication capabilities. A third-party server 140 may be a Hypertext Transfer Protocol (HTTP) server, a Representational State Transfer (REST) service, or other server type, having structure and/or functionality for processing and satisfying content requests and/or requesting and receiving content from one or more of the client devices 115, the data sources 135, and the healthcare management server 120 that are coupled to the network 105. In some implementations, the third-party server 140 may include an online service 111 dedicated to providing access to various services and information resources hosted by the third-party server 140 via web, mobile, enterprise, and/or cloud applications. The online service 111 may obtain and store user data, user-generated data, content items (e.g., videos, text, images, etc.), and interaction data reflecting the interaction of users with the content items. In some implementations, the third-party server 140 may provide an API 136 to facilitate access of the third-party server 140 by one or more of the client devices 115, the data sources 135, and the healthcare management server 120 that are coupled to the network 105. User-generated data, as described herein, may include one or more of user profile information (e.g., user id, user preferences, user history, social network connections, primary care physicians, etc.), logged information (e.g., heart rate, activity metrics, sleep quality data, calories and nutrient data, user device specific information, historical actions, medication history, etc.), and other user specific information. In some implementations, the online service 111 allows users to share content with other users (e.g., friends, contacts, public, similar users, primary care physicians, clinical staff, administrative staff, etc.), purchase and/or view items (e.g., e-books, videos, music, games, subscription, fitness products, prescription refill, laboratory results, etc.), and other similar actions. For example, the online service 111 may provide various services such as digital fitness content; personal training; running and cycling tracking service; music streaming service; mobile health (mHealth) service; video streaming service; web mapping service; multimedia messaging service; electronic mail service; a calendar service; news service; news aggregator service; social networking service; location-based service; photo and video-sharing social networking service; sleep-tracking service; diet-tracking and calorie counting service; ridesharing service; online banking service; online information database service; travel service; online e-commerce marketplace; ratings and review service; restaurant-reservation service; food delivery service; search service; health and fitness service; home automation and security service; Internet of Things (IOT), multimedia hosting, distribution, and sharing service; cloud-based data storage and sharing service; a scheduling service; an enterprise clinical workflow service; a combination of one or more of the foregoing services; or any other service where users retrieve, collaborate, and/or share information, etc. It should be noted that the list of items provided above as examples for the online service 111 above are not exhaustive and that others are contemplated in the techniques described herein.

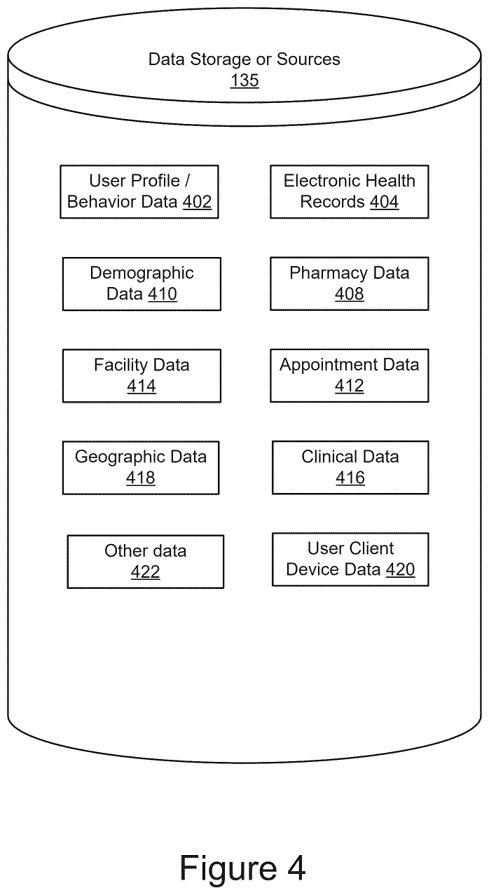

Each of the plurality of data sources 135 may be, or may be implemented by, a computing device including a processor, a memory, applications, a database, and network communication capabilities. In some implementations, the data sources may be a data warehouse, a system of record (SOR), or belonging to a data repository owned by an organization that provides real-time or close to real-time data automatically or responsive to being polled or queried by the healthcare management server 120. Each of the plurality of data sources 135 may be associated with a first-party entity (e.g., server 120) or third-party entity (e.g., server 140 associated with a separate company or service provider), such as a health insurance organization, a health care organization, world health organization, an independent healthcare provider, a healthcare-related call center or customer service company, a healthcare software company, an Electronic Medical Record (EMR) software company, an Electronic Health Record (EHR) software company, a pharmacy management system, a drug research institute, a patient management software system, a clinical decision support system, a clinical workflow management system, a scheduling system, a patient-satisfaction measurement firm, a medication adherence tracking system, a public-records database, a data mining platform, a Software as a Service (SaaS) data analytics company, a data science and machine learning platform, news site, support groups, health blogs, etc. Examples of data provided by the plurality of data sources 135 may include, but is not limited to, pharmacy data, physician-patient encounter data, clinical data, patient data, EMR, EHR, patient diagnosis data, patient procedures, appointment notes, socioeconomic data, social determinant data, demographic data, health plan data, prescription data, call center data, appointment schedule data, disposition data, calendar data, medication data, pharmaceutical data, survey data, medication adherence data, machine learning models, machine learning-based data analysis results, etc. In some implementations, each of the plurality of data sources 135 may be configured to provide or facilitate an API (not shown) that allows the conversational care optimization module 110 to access data and information for performing the functionality described herein. The data storage or sources 135 will be described in more detail below with reference to FIG. 4.

The conversational care optimization module 110 may include software and/or logic to provide the functionality for optimizing care in a conversational manner for delivering the appropriate healthcare services. In some implementations, the conversational care optimization module 110 may be implemented using programmable or specialized hardware, such as a field-programmable gate array (FPGA) or an application-specific integrated circuit (ASIC). In some implementations, the conversational care optimization module 110 may be implemented using a combination of hardware and software. In one implementation, the conversational care optimization module 110b is stored and executed on healthcare management server 120 alone. In another implementation (not pictured), the conversational care optimization module 110a . . . 110n is stored and executed on client device 115 alone. In other implementations, the conversational care optimization module 110 may be stored and executed on various combinations of the client device 115, the data sources 135, the third-party servers 140, and the healthcare management server 120.

In some implementations, the conversational care optimization module 110 may be a thin-client application with some functionality executed on the client device 115 and additional functionality executed on the healthcare management server 120 by the conversational care optimization module 110. In some implementations, the conversational care optimization module 110 may generate and present various user interfaces to perform these acts and/or functionality, which may in some cases be based at least in part on information received from the healthcare management server 120, the client device 115, one or more of the third-party servers 140 and/or the data sources 135 via the network 105. In some implementations, the conversational care optimization module 110 is code operable in a web browser, a web application accessible via a web browser, a native application (e.g., mobile application, installed application, etc.) on the client device 115, a combination thereof, etc. Additional structure, acts, and/or functionality of the conversational care optimization module 110 is further discussed below with reference to at least FIG. 2.

In some implementations, the conversational care optimization module 110 may require users to be registered with the healthcare management server 120 to access the actions and/or functionality described herein. For example, to access various actions and/or functionality provided by the conversational care optimization module 110 may require a user to authenticate their identity. For example, the conversational care optimization module 110 may require a user seeking access to authenticate their identity by inputting credentials in an associated user interface. In another example, the conversational care optimization module 110 may interact with a federated identity server (not shown) to register and/or authenticate the user by scanning and verifying biometrics including username and password, facial attributes, fingerprint, and voice.

The automatic medication reconciliation module 112 may include software and/or logic to provide the functionality for automatic medication reconciliation for delivering the appropriate healthcare services. For example, a list of prescription medications may be maintained in data storage or sources 135 and may be accessed by the healthcare management server 120. Additionally, the conversational care optimization module 110 may operate in conjunction with the automatic medication reconciliation module 112 to present information to the user 106 through a client device 115 in a conversational user interface or application. In this way, the medication reconciliation process may be made automatic in that the user is prompted through a conversational user interface about the various medications they are taking, how regularly they are taking the medications, what symptoms or side effects they are experiencing in conjunction with different medications, and other issues related to patient care. In this way, the patient member user may take their time in reviewing their medications and better outcomes may result by using the automatic medication reconciliation process implemented by the automatic medication reconciliation module 112. As shown in FIG. 1, the automatic medication reconciliation module 112a . . . 112n may operate on client devices 115a . . . 115n through a web browser, a mobile application, or any other type of software logic executable on the client device 115.

Other variations and/or combinations are also possible and contemplated. It should be understood that the system 100 illustrated in FIG. 1 is representative of an example system and that a variety of different system environments and configurations are contemplated and are within the scope of the present disclosure. For example, various acts and/or functionality may be moved from a server 120 to a client device 115, or vice versa, data may be consolidated into a single data store or further segmented into additional data stores, and some implementations may include additional or fewer computing devices, services, and/or networks, and may implement various functionality client or server-side. Furthermore, various entities of the system may be integrated into a single computing device or system or divided into additional computing devices or systems, etc.

FIG. 2 is a block diagram illustrating one implementation of a computing device 200 including the conversational care optimization module 110 and the automatic medication reconciliation module 112. The computing device 200 may also include a processor 235, a memory 237, a display device 239, a communication unit 241, an input/output device(s) 247, and a data storage 135, according to some examples. The components of the computing device 200 are communicatively coupled by a bus 220. In some implementations, the computing device 200 may be representative of the client device 115, the healthcare management server 120, or a combination of the client device 115 and the healthcare management server 120. In such implementations where the computing device 200 is the client device 115 or the healthcare management server 120, it should be understood that the client device 115 and the healthcare management server 120 may take other forms and include additional or fewer components without departing from the scope of the present disclosure. For example, while not shown, the computing device 200 may include sensors, capture devices, additional processors, and other physical configurations. Additionally, it should be understood that the computer architecture depicted in FIG. 2 could be applied to other entities of the system 100 with various modifications, including, for example, the servers 140 and data sources 135.

The processor 235 may execute software instructions by performing various input/output, logical, and/or mathematical operations. The processor 235 may have various computing architectures to process data signals including, for example, a complex instruction set computer (CISC) architecture, a reduced instruction set computer (RISC) architecture, and/or an architecture implementing a combination of instruction sets. The processor 235 may be physical and/or virtual, and may include a single processing unit or a plurality of processing units and/or cores. In some implementations, the processor 235 may be capable of generating and providing electronic display signals to a display device 239, supporting the display of images, capturing and transmitting images, and performing complex tasks including various types of feature extraction and sampling. In some implementations, the processor 235 may be coupled to the memory 237 via the bus 220 to access data and instructions therefrom and store data therein. The bus 220 may couple the processor 235 to the other components of the computing device 200 including, for example, the memory 237, the communication unit 241, the display device 239, the input/output device(s) 247, and the data storage 135. Additionally, the computing device 200 may optionally include sensors 249 and a capture device 245.

The memory 237 may store and provide access to data for the other components of the computing device 200. The memory 237 may be included in a single computing device or distributed among a plurality of computing devices as discussed elsewhere herein. In some implementations, the memory 237 may store instructions and/or data that may be executed by the processor 235. The instructions and/or data may include code for performing the techniques described herein. For example, as depicted in FIG. 2, the memory 237 may store the conversational care optimization module 110 and the automatic medication reconciliation module 112. The memory 237 is also capable of storing other instructions and data, including, for example, an operating system, hardware drivers, other software applications, databases, etc. The memory 237 may be coupled to the bus 220 for communication with the processor 235 and the other components of the computing device 200.

The memory 237 may include one or more non-transitory computer-usable (e.g., readable, writeable) device, a static random access memory (SRAM) device, a dynamic random access memory (DRAM) device, an embedded memory device, a discrete memory device (e.g., a PROM, FPROM, ROM), a hard disk drive, an optical disk drive (CD, DVD, Blu-ray™, etc.) mediums, which can be any tangible apparatus or device that can contain, store, communicate, or transport instructions, data, computer programs, software, code, routines, etc., for processing by or in connection with the processor 235. In some implementations, the memory 237 may include one or more of volatile memory and non-volatile memory. It should be understood that the memory 237 may be a single device or may include multiple types of devices and configurations.

The bus 220 may represent one or more buses including an industry standard architecture (ISA) bus, a peripheral component interconnect (PCI) bus, a universal serial bus (USB), or some other bus providing similar functionality. The bus 220 may include a communication bus for transferring data between components of the computing device 200 or between computing device 200 and other components of the system 100 via the network 105 or portions thereof, a processor mesh, a combination thereof, etc. In some implementations, the conversational care optimization module 110 and various other software operating on the computing device 200 (e.g., an operating system, device drivers, etc.) may cooperate and communicate via a software communication mechanism implemented in association with the bus 220. The software communication mechanism may include and/or facilitate, for example, inter-process communication, local function or procedure calls, remote procedure calls, an object broker (e.g., CORBA), direct socket communication (e.g., TCP/IP sockets) among software modules, UDP broadcasts and receipts, HTTP connections, etc. Further, any or all of the communication may be configured to be secure (e.g., SSH, HTTPS, etc.).

The display device 239 may be any conventional display device, monitor or screen, including but not limited to, a liquid crystal display (LCD), light emitting diode (LED), organic light-emitting diode (OLED) display or any other similarly equipped display device, screen or monitor. The display device 239 represents any device equipped to display user interfaces, electronic images, and data as described herein. In some implementations, the display device 239 may output display in binary (only two different values for pixels), monochrome (multiple shades of one color), or multiple colors and shades. The display device 239 is coupled to the bus 220 for communication with the processor 235 and the other components of the computing device 200. In some implementations, the display device 239 may be a touch-screen display device capable of receiving input from one or more fingers of a user. For example, the display device 239 may be a capacitive touch-screen display device capable of detecting and interpreting multiple points of contact with the display surface. In some implementations, the computing device 200 (e.g., client device 115) may include a graphics adapter (not shown) for rendering and outputting the images and data for presentation on display device 239. The graphics adapter (not shown) may be a separate processing device including a separate processor and memory (not shown) or may be integrated with the processor 235 and memory 237.

The input/output (I/O) device(s) 247 may include any standard device for inputting or outputting information and may be coupled to the computing device 200 either directly or through intervening I/O controllers. In some implementations, the input device 247 may include one or more peripheral devices. Non-limiting example I/O devices 247 include a touch screen or any other similarly equipped display device equipped to display user interfaces, electronic images, and data as described herein, a touchpad, a keyboard, a scanner, a stylus, an audio reproduction device (e.g., speaker), a microphone array, a barcode reader, an eye gaze tracker, a sip-and-puff device, and any other I/O components for facilitating communication and/or interaction with users. In some implementations, the functionality of the input/output device 247 and the display device 239 may be integrated, and a user of the computing device 200 (e.g., client device 115) may interact with the computing device 200 by contacting a surface of the display device 239 using one or more fingers. For example, the user may interact with an emulated (i.e., virtual or soft) keyboard displayed on the touch-screen display device 239 by using fingers to contact the display in the keyboard regions.

The communication unit 241 is hardware for receiving and transmitting data by linking the processor 235 to the network 105 and other processing systems via signal line 104. The communication unit 241 receives data such as message from the client device 115 and transmits the message to the conversational care optimization module 110, for example, an email, a post, a text, website input, a mobile application, a phone call, interactive voice response (IVR), instant messaging chat, etc. The message may provide information about a condition or request for service from a healthcare provider. The communication unit 241 also transmits information including media to the client device 115 for display, for example, in response to the request. The communication unit 241 is coupled to the bus 220. In some implementations, the communication unit 241 may include a port for direct physical connection to the client device 115 or to another communication channel. For example, the communication unit 241 may include an RJ45 port or similar port for wired communication with the client device 115. In other implementations, the communication unit 241 may include a wireless transceiver (not shown) for exchanging data with the client device 115 or any other communication channel using one or more wireless communication methods, such as IEEE 802.11, IEEE 802.16, Bluetooth® or another suitable wireless communication method.

In yet other implementations, the communication unit 241 may include a cellular communications transceiver for sending and receiving data over a cellular communications network such as via short messaging service (SMS), multimedia messaging service (MMS), hypertext transfer protocol (HTTP), direct data connection, WAP, e-mail or another suitable type of electronic communication. In still other implementations, the communication unit 241 may include a wired port and a wireless transceiver. The communication unit 241 also provides other conventional connections to the network 105 for distribution of files and/or media objects using standard network protocols such as TCP/IP, HTTP, HTTPS, and SMTP as will be understood to those skilled in the art.

The data storage 135 is a non-transitory memory that stores data for providing the functionality described herein. In some implementations, the data storage 135 may be coupled to the components 235, 237, 239, 241, 135, and 247 via the bus 220 to receive and provide access to data. In some implementations, the data storage 135 may store data received from other elements of the system 100 including, for example, entities 135, 140, and/or the conversational care optimization module 110, and may provide data access to these entities. The data storage 135 may store, among other data, user profiles, patient medical data, training datasets, machine learning models, and one or more metadata stores. The data stored in the data storage 135 is described below in more detail with reference to FIG. 4.

The data storage 135 may be included in the computing device 200 or in another computing device and/or storage system distinct from but coupled to or accessible by the computing device 200. The data storage 135 may include one or more non-transitory computer-readable mediums for storing the data. In some implementations, the data storage 135 may be incorporated with the memory 237 or may be distinct therefrom. The data storage 135 may be a dynamic random-access memory (DRAM) device, a static random-access memory (SRAM) device, flash memory, or some other memory devices. In some implementations, the data storage 135 may include a database management system (DBMS) operable on the computing device 200. For example, the DBMS could include a structured query language (SQL) DBMS, a NoSQL DMBS, various combinations thereof, etc. In some instances, the DBMS may store data in multi-dimensional tables comprised of rows and columns, and manipulate, e.g., insert, query, update and/or delete, rows of data using programmatic operations. In other implementations, the data storage 135 also may include a non-volatile memory or similar permanent storage device and media including a hard disk drive, a CD-ROM device, a DVD-ROM device, a DVD-RAM device, a DVD-RW device, a flash memory device, or some other mass storage device for storing information on a more permanent basis.

It should be understood that other processors, operating systems, sensors, displays, and physical configurations are possible.

As depicted in FIG. 2, the memory 237 may include the conversational care optimization module 110. In some implementations, the conversational care optimization module 110 may be configured to implement a secure HTTP API (not shown) to facilitate receipt and transmission of messages via the web, mobile, enterprise, and/or cloud applications for providing patients with access to care for appropriate healthcare services. Similarly, the automatic medication reconciliation module 112 may be configured to send and receive messages through the same or different secure HTTP API.

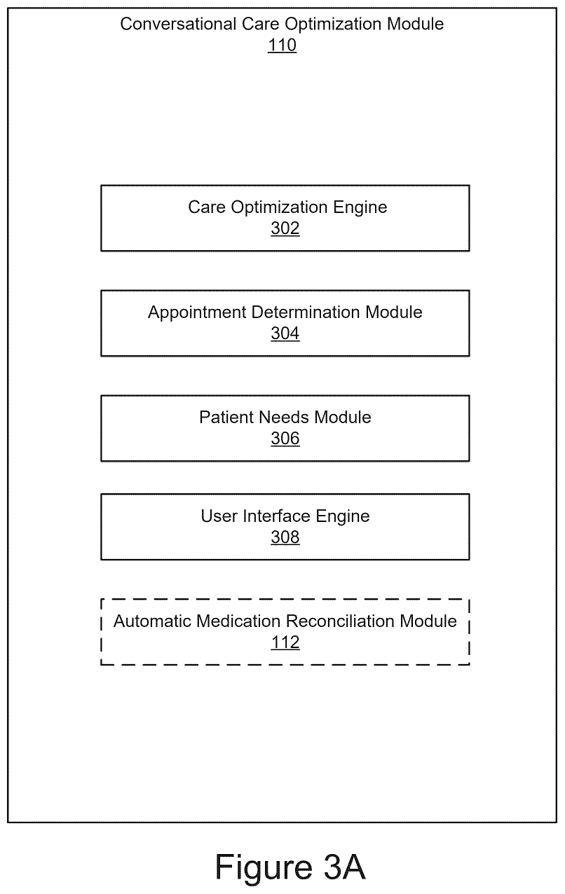

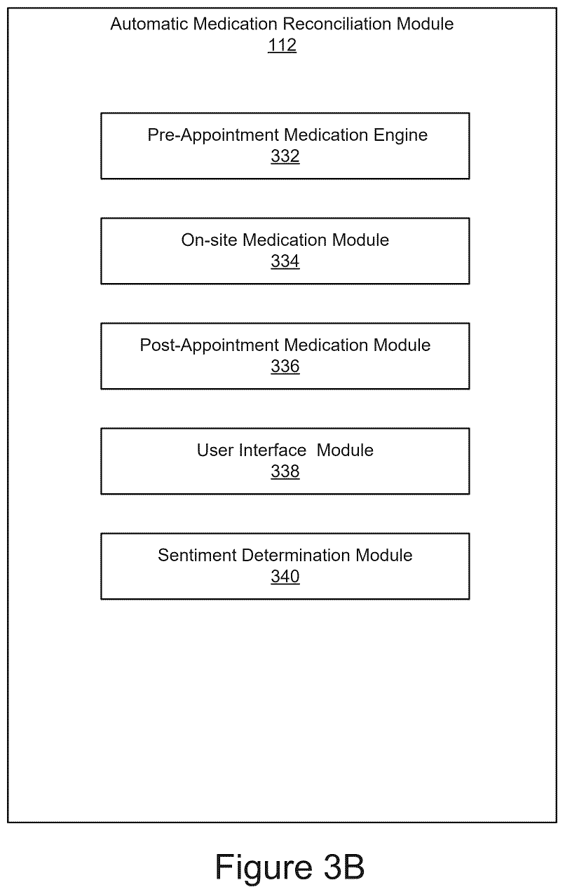

In some implementations, the conversational care optimization module 110 may include a care optimization engine 302, an appointment determination module 304, a patient needs module 306, a user interface engine 308 and, optionally, the automatic medication reconciliation module 112. These components and their operation will be described in more detail below with reference to FIG. 3A. The components 302, 304, 306, 308, and 112 may be communicatively coupled by the bus 220 and/or the processor 235 to one another and/or the other components 237, 239, 241, 135, and 247 of the computing device 200 for cooperation and communication. The components 302, 304, 306, 308, and 112 may each include software and/or logic to provide their respective functionality. In some implementations, the components 302, 304, 306, 308, and 112 may each be implemented using programmable or specialized hardware including a field-programmable gate array (FPGA) or an application-specific integrated circuit (ASIC). In some implementations, the components 302, 304, 306, 308, and 112 may each be implemented using a combination of hardware and software executable by the processor 235. In some implementations, each one of the components 302, 304, 306, 308, and 112 may be sets of instructions stored in the memory 237 and configured to be accessible and executable by the processor 235 to provide their acts and/or functionality. In some implementations, the components 302, 304, 306, 308, and 112 may send and receive data, via the communication unit 241, to and from one or more of the client devices 115, the healthcare management server 120, the data sources 135, and third-party servers 140. The operation and interaction of the care optimization engine 302, the appointment determination module 304, the patient needs module 306, the user interface engine 308 and the automatic medication reconciliation module 112 will be described in more detail below with reference to FIGS. 3A-3B and 5-9.

Referring now to FIG. 3A, one implementation for the conversational care optimization module 110 is described. In some implementations, the conversational care optimization module 110 includes a care optimization engine 302, an appointment determination module 304, a patient needs module 306, a user interface engine 308 and, optionally, the automatic medication reconciliation module 112. In some implementations, each of these 302, 304, 306, 308, and 112 are coupled for communication with each other, other components of the computing system 200, and other components of the system 100 for providing improved medical care by optimizing patients' pre- and post-visit actions. Although described below primarily as an application that optimizes pre- and post-visit actions by the patient, in some implementations, the conversational care optimization module 110 effectively operates as an application that nudges the patient by sending them various types of communications (email, text messages, etc.) based on the context of the patient. Any variety of information can be used to define a patient's context, including but not limited to, the patient's medical record (e.g., do they have any upcoming appointments, any open lab orders, conditions, etc.), location (e.g., hospitals, clinics, testing locations, etc.) and time (time of day, day of week, month, year, etc.), medical record (e.g., do they have any upcoming appointments, any open lab orders, etc.), location (e.g., geographic) and time (day and time of day).

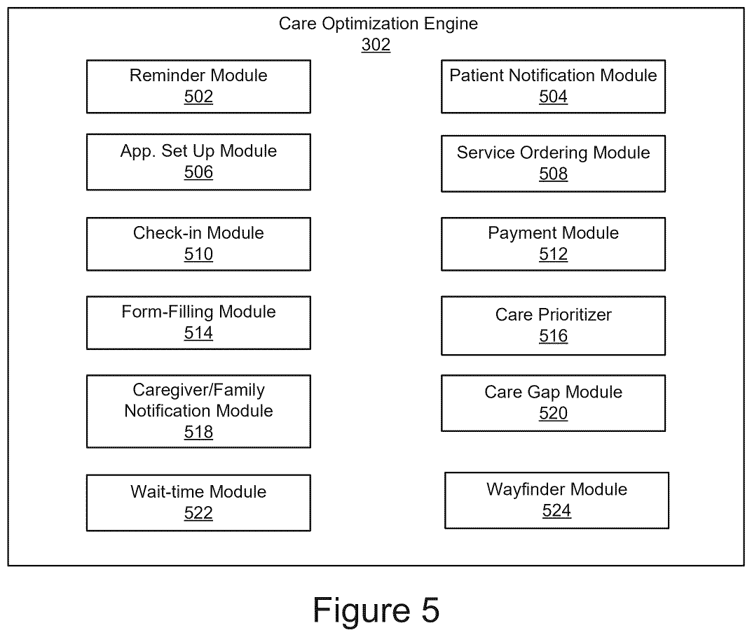

The care optimization engine 302 may include software and/or logic for identifying one or more actions that a user/patient can perform outside of a visit to a facility. The care optimization engine 302 is coupled to the appointment determination module 304, the patient needs module 306, the user interface engine 308 and, optionally, the automatic medication reconciliation module 112, to receive information for determining the one or more actions to present to a user outside of their visit to a facility. The care optimization engine 302 determines the follow up actions that need to be performed, the patient's needs for other medical services, and the particular action that will most optimize the patient's health. One example implementation for the care optimization engine 302 is described in more detail below with reference to FIG. 5. The care optimization engine 302 generates a recommendation for optimization of actions to be performed by a user after their visit to a facility. The recommendation is provided to the user via the user interface engine 308 for presentation to the user through a client device 115, for example. One or more actions can be included depending on information such as one or more prescriptions given after a visit, one or more tests recommended after a visit, one or more referrals for additional care that a patient may have requested at the visit, educational information related to the visit, and ordering information for durable medical equipment after the visit, as identified by the patient needs module 306.

The appointment determination module 304 may include software and/or logic for determining an upcoming appointment for a given patient. For example, a list of upcoming appointments that one or more providers have scheduled on the patient's behalf may be presented to the user on a client device 115 through the appointment determination module 304. Each appointment may have an expected date of completion as well as an expiration date, in an embodiment. The appointment determination module 304 can also be used to determine other services that have availability after an appointment for a given patient. The appointment determination module 304 is coupled to the user interface engine 308 to determine a particular user for which to find a schedule appointment or to schedule an appointment for the user. The appointment determination module 304 is coupled to the care optimization engine 302 to determine a priority of different services offered at different facilities, based on date and other factors. The appointment determination module 304 provides information about appointments that are available to the patient based on their needs to the care optimization engine 302. In an embodiment, the appointment determination module 304 only provides patient information to the patient and to no other proxies (e.g., caregivers, parents, guardians, or other family members or designated individuals) to respect the privacy of the patient. For example, a teenage patient member user may desire to go on birth control medication and may not wish to have that information disclosed to their parents.

The patient needs module 306 may include software and/or logic for determining particular care and/or services needed by a patient. Those services can be determined based upon the medical condition of the patient, recommendations by physicians for that patient, the preferred standard of a care for patients with particular medical conditions, services based upon symptoms of a patient, or services desired by a patient. The patient needs module 306 may be coupled to other third-party servers 140 and/or the data storage 135 to receive information for making recommendations about which medical providers to suggest for the patient. In some implementations, some or all of the functionality provided by the patient needs module 308 may be incorporated into the care optimization engine 302. The patient needs module 306 is coupled to provide recommendations for patient care to the care optimization engine 302. The recommendations provided by the patient needs module 306 can include prioritization associated with particular recommendations. For example, more serious symptomatic conditions may come with recommendations that have a higher prioritization than day-to-day preventive care recommendations. In an embodiment, referrals provided by a health care provider's office may be retrieved and provided for display to a patient by the patient needs module 306 through a user interface engine 308, in an embodiment. Information about each referral may be retrieved and provided for display, including a referral number, a medical provider referred to, a medical provider referred by, a start date, an expiration date, and a status (authorized, canceled, closed, or pending review). For example, a general practitioner may provide a referral to a specialist to treat a specific condition, such as a dermatologist for treating a specific skin condition. In this way, the referrals may be organized and tracked through a single user interface, improving the user experience and improving patient care. Additionally, notifications may be generated based on a status of the referral, such as whether the referral has been authorized by insurance, for example, or when the expiration date is within a certain amount of time. In a further embodiment, patient needs related to medications, such as interactions with medications, adverse reactions such as nausea, stomach ache, pains, and so forth, may be captured through the conversational user interface and stored as patient needs data in the data storage or sources 135 by the patient needs module 306.

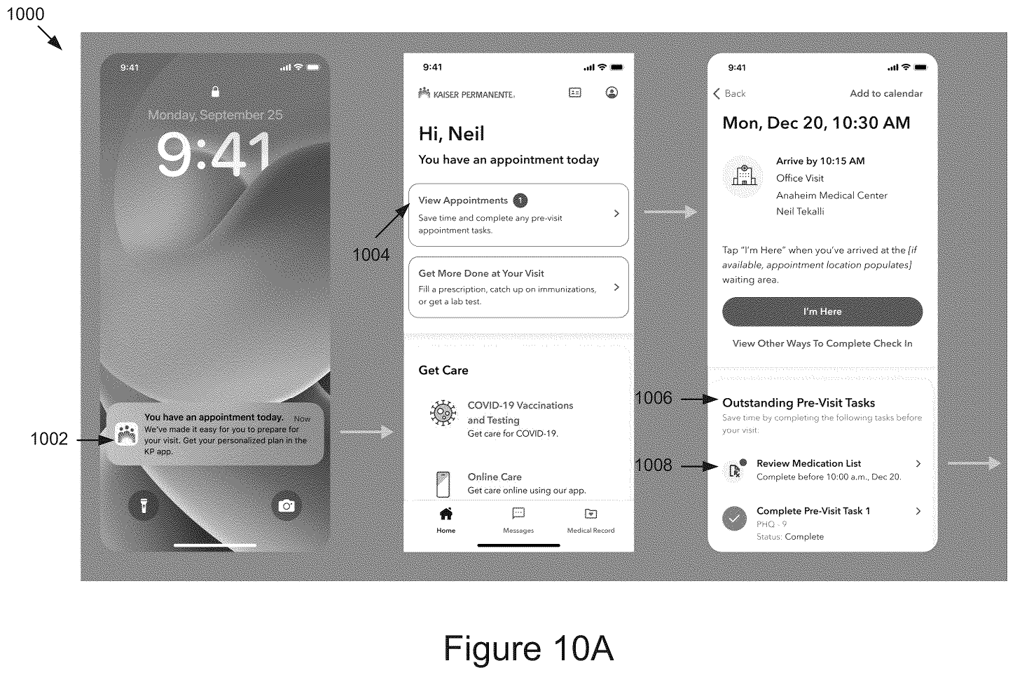

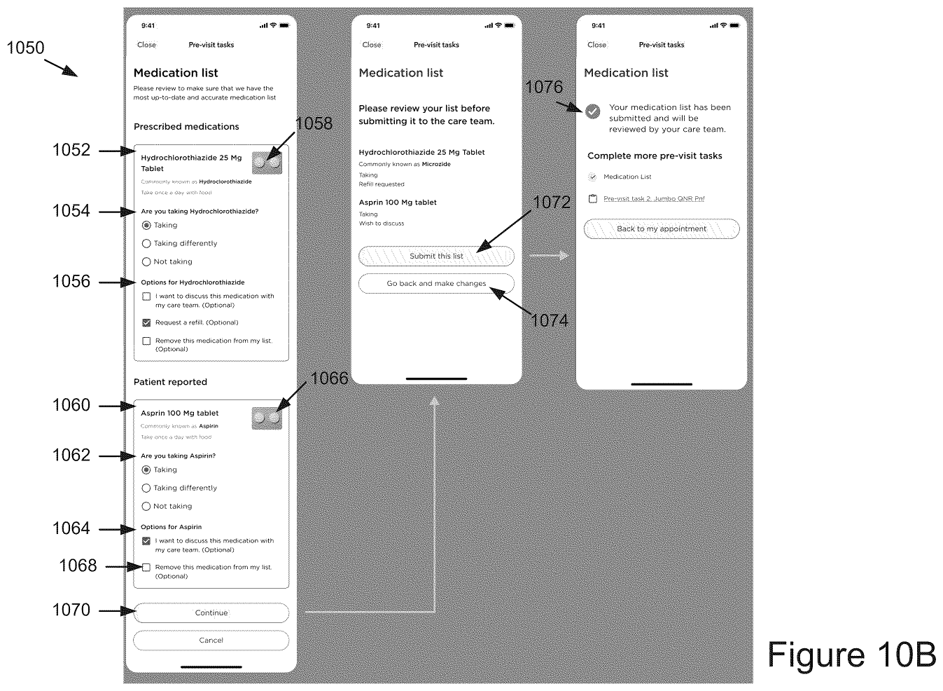

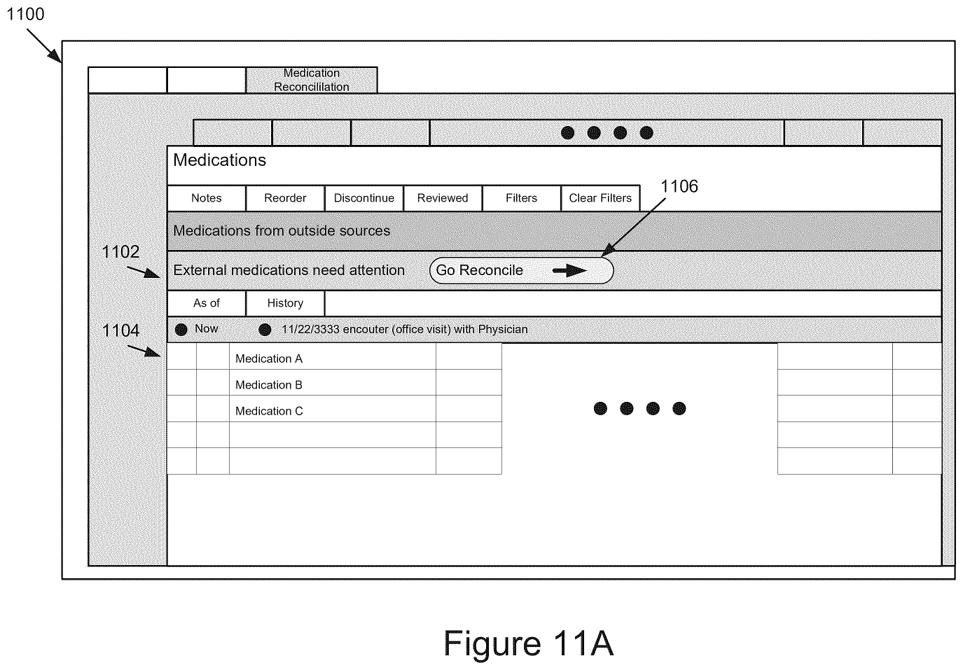

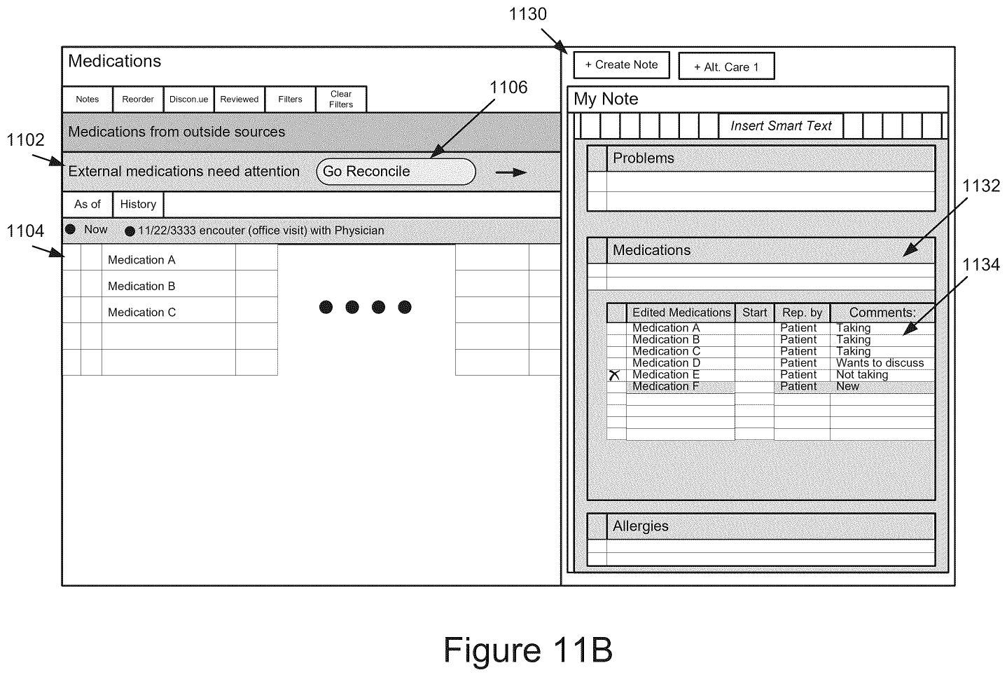

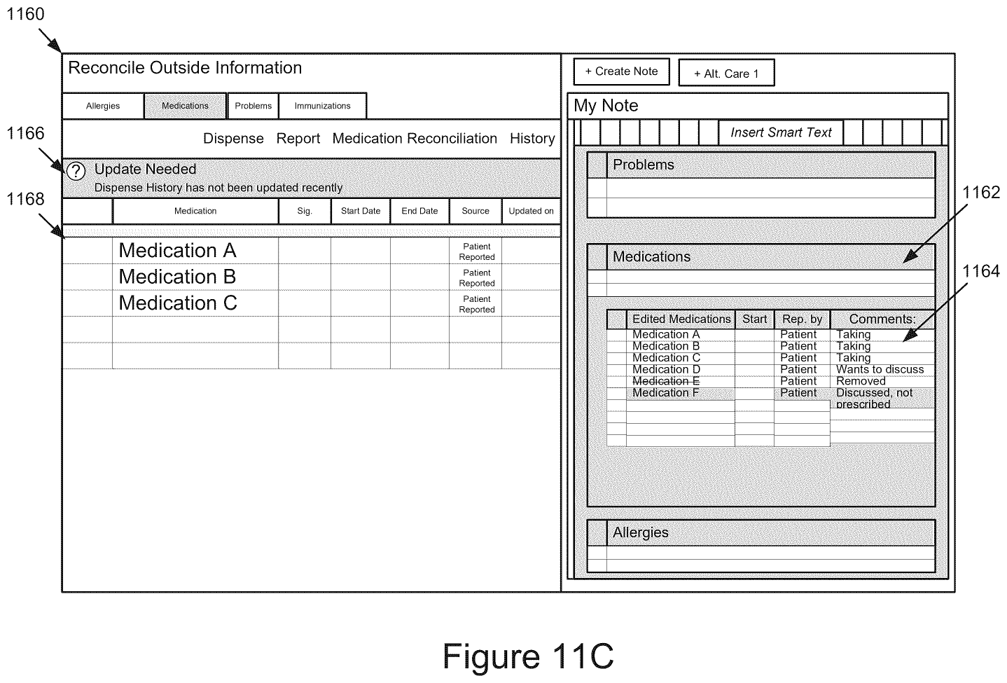

The user interface engine 308 may include software and/or logic for providing user interfaces to a user or patient. In some implementations, the user interface engine 308 receives instructions from the components 302, 304, 306, and 112, generates a user interface according to the instructions, and transmits the user interface for display on the client device 115. In some implementations, the user interface engine 308 sends graphical user interface data to an application (e.g., a browser) in the client device 115 via the communication unit 241 causing the application to display the data as a graphical user interface. In some implementations, the user interface engine 308 also receives input from the user via the client device 115. The user interface engine 308 is coupled to provide user input to the other components 302, 304, 306, and 112 of the conversational care optimization module 110. Example user interfaces generated by the user interface engine 308 will be shown and described in more detail below with reference to FIGS. 10A-10B and 11A-11C.

In some implementations, the conversational care optimization module 110 may also be configured to provide optimize treatment or services at other locations such as the patient's home, consumer locations, ride-share services, non-medical services or testing offices, etc. For example, the conversational care optimization module 110 may also optimize or suggest other post-surgical services like ride-sharing to and from a facility, remote health care monitoring, off-site care delivery, asset (medical equipment) tracking, etc. In another example, the conversational care optimization module 110 may also get and coordinate the delivery of other services such as physical assistance, a wheel chair (e.g., for a senior, an expecting mom, any disabled patient), or any other types of assistance that a patient may need from when the leave a medical facility. In yet another example, the conversational care optimization module 110 may use geolocation information to find or suggest the closest provider, physician, or facility to provide after-visit care that a patient needs. The conversational care optimization module 110 may find the closest facility, pharmacy, ER, and can find closest doctor and location when scheduling post-visit care actions and can be integrated to virtual urgent care decision making. In still another example, the conversational care optimization module 110 may manage and assist in post-surgical care for a patient. The conversational care optimization module 110 may use the patient's geolocation signal to track recovery (steps walked) and any assets such as durable medical equipment sent home with the patient after the procedure (walker, ventilators etc.), and schedule post-surgical follow ups or find emergency facilities depending the patient's recovery. In an embodiment, the conversational care optimization module 110 may receive information items from third-party servers 140 that provide an online service 111 to perform one or more of the above functions. Additionally, these information items may be stored in data storage or sources 135 accessible by the conversational care optimization module 110 through network 105. In this way, various actions may be prioritized and/or optimized by the conversational care optimization module 110 based on the information received from third-party servers 140 and/or retrieved from data storage or sources 135. This beneficially optimizes delivery of health care and provides an improved health care user experience.

The automatic medication reconciliation module 112 may include software and/or logic for gathering and providing information related to medication reconciliation via user interfaces accessed by a user or patient. Traditional medication reconciliation involves a primary care provider asking a patient about the medications being currently taken by the patient. Oftentimes, the patient may have forgotten their prescription bottles at home or may not remember their adherence to a particular medication. Thus, the automatic medication reconciliation module 112 enables software and/or logic for a user interface to be presented to the patient member user to be prompted to fill out a form or respond to a conversational user interface regarding specific medications, such as “Are you taking this medication as prescribed?” and “How regularly are you taking this medication?” as well as other questions regarding current usage or other patient behavior around medication. Additionally, pictures of the pills may be presented to the member user or patient to refresh the patient's memory. In another embodiment, for each medication, a question such as “Are you taking Hydrochlorothiazide?” with radio buttons for “Taking” and “Not taking” presented in a form, as shown in FIG. 10B, that forces the user to select only one option. In a further embodiment, another radio button may be presented, such as “taking differently than prescribed.” In yet another embodiment, other user interface elements may be presented, such as “I want to discuss Hydrochlorothiazide with my care team” as a checkbox and “Request a refill of Hydrochlorothiazide” as another checkbox, where both checkboxes may be optional. Additionally, a patient may self-report other medications, drugs, vitamins and/or supplements they are taking. For example, a patient may have previously reporting taking Vitamin B, so that the form may request the patient member user to respond whether they are taking or not taking the self-reported medications, drugs, vitamins and/or supplements and whether the patient member would like to discuss a particular item (optionally) with a care professional. As a result of the user filling out the form for each medication, the patient data is updated in data storage or sources 135.

The automatic medication reconciliation module 112 also provides software and/or logic to provide the physician experience of viewing the patient-reported data. As shown in FIGS. 11A-11C, a notification may alert the physician or primary care provider indicating that medication reconciliation needs to be performed. The data provided by the patient or user, through the user interface providing a form or a conversational user interface, is presented to the physician or primary care provider in a tabular format or other presentation method to show the list of medications, the suggested medication adherence, a start date, an end date, source, and last updated date. Additionally, a note may be created by the physician or primary care provider during the medication reconciliation with the patient, such as adding a note on who reports the medication adherence (e.g., patient, spouse, or other caregiver) as well as comments (Taking, Wants to discuss, not taking, other) for each medication.

FIG. 3B illustrates an example implementation of the automatic medication reconciliation module 112. In some implementations, the automatic medication reconciliation module 112 may include a pre-appointment medication engine 332, an on-site medication module 334, a post-appointment medication module 336, a user interface module 338, and a sentiment determination module 340. In some implementations, each of these 332, 334, 336, 338, and 340 are coupled for communication with each other, other components of the computing system 200, and other components of the system 100 for providing improved medical care by optimizing patients' pre- and post-visit actions. Although described below primarily as an application that optimizes pre- and post-visit actions by the patient, in some implementations, the conversational care optimization module 110 effectively operates as an application that nudges the patient by sending them various types of communications (email, text messages, etc.) based on the context of the patient. Any variety of information can be used to define a patient's context, including but not limited to, the patient's medical record (e.g., do they have any upcoming appointments, any open lab orders, conditions, etc.), location (e.g., hospitals, clinics, testing locations, etc.) and time (time of day, day of week, month, year, etc.), medical record (e.g., do they have any upcoming appointments, any open lab orders, etc.), location (e.g., geographic) and time (day and time of day).

The pre-appointment medication engine 332 may include software and/or logic for storing, sending, and retrieving data attributes related to medications that have been previously prescribed to a patient. The pre-appointment medication engine 332 enables medication data to be retrieved from data storage or sources 135 and populates a user interface with the list of medications for the patient and/or care provider. The pre-appointment medication engine 332 further includes functionality for sending a reminder to the patient via a conversational interface application to complete the pre-appointment task of completing the medication review form or otherwise engaging with the conversational interface application. After completion of the medication review process via the conversational interface application, the pre-appointment medication engine 332 sends a message to the modules of the healthcare management server 120 indicating the updated medication data.

The on-site medication module 334 may include software and/or logic for providing an interface for a care provider to interact with the received medication data. A client device for a care provider may include a desktop computer running an enterprise software application or other application connected to a third-party service or server. The on-site medication module 334 enables the care provider to interact with and update the list of medications through the medication reconciliation process in a more automated manner than traditional medication review. For example, a patient may wish to discuss a new medication, such as birth control, where the patient is underage. The patient may indicate this desire to discuss birth control through the conversational interface application as described herein. A parent or guardian may not have access to the application, in an embodiment. This beneficially improves the heath care delivered to the patient. As another example, a patient may want to discuss an alternative medication to the medication currently prescribed by the care provider. The on-site medication module 334 may retrieve information related to the requested medication and/or alternatives. The on-site medication module 334 may also perform care optimizations such as suggesting that the care provider alter dosages, change or delete medications, and/or order refills for the patient through the client device for the care provider. As a result, care optimizations may be performed in real-time through the on-site medication module 334.

The post-appointment medication module 336 may include software and/or logic for updating and/or confirming medication data with the patient after an appointment. In some implementations, one or more care optimizations may be generated based on the medication data received before and during the appointment. For example, a machine learning model may be used to identify alternative medications that may be better suited to a patient given the symptoms they are experiencing while using a prescribed medication. As a result of notes taken by a care provider and/or messages received from a patient in response to taking a new medication, the medication data may be analyzed and contribute to generating one or more care optimizations, as an example. The post-appointment medication module 336 may also store the updated medication data once confirmed by the patient through the conversational interface application.

The user interface module 338 may include software and/or logic for providing user interfaces to a care provider. In some implementations, the user interface module 338 receives instructions from the components 332, 334, 336, and 340, generates a user interface according to the instructions, and transmits the user interface for display on the client device 115. In some implementations, the user interface module 338 sends graphical user interface data to an application (e.g., a browser) in the client device 115 via the communication unit 241 causing the application to display the data as a graphical user interface. In some implementations, the user interface module 338 also receives input from the user via the client device 115. The user interface module 338 is coupled to provide user input to the other components 332, 334, 336, and 340 of the automatic medication reconciliation module 112. Example user interfaces generated by the user interface module 338 will be shown and described in more detail below with reference to FIGS. 10A-10B and 11A-11C.

The sentiment determination module 340 may include software and/or logic for generating a sentiment determination based on user feedback for surveys regarding medication review. In an embodiment, certain patients, such as MEDICARE recipients, may be asked survey questions related to how often their care providers engaged in medication review. The sentiment determination module 340 may enable analysis of the responses and other user feedback to make changes to the medication reconciliation process, such as adding additional reminders for completing medication review before a visit as well as after a visit to confirm changes.

FIG. 4 is a block diagram illustrating an example implementation of the data storage or sources 135. In some implementations, the data storage or sources 135 may include user profile/behavior data 402, electronic health records 404, pharmacy data 408, demographic data 410, appointment data 412, facility data 414, clinical data 416, geographic data 418, user client device data 420, and other data 422. Although shown as a single data storage in FIG. 4, it should be understood that the data storage or sources 135 may be a plurality of different data storage devices. Each of the types of data described above may be stored in various combinations in different configurations of data storage devices. Additionally, the different portions of data may be distributed across the system 100, and associated with different third-party servers 140. The configuration shown in FIG. 1 and FIG. 4 are provided merely by way of example. Each of the datatypes may be accessed by different components of the conversational care optimization module 110. Additionally, the description of the data types below is merely by way of example. Each of the data types may include more or less data, data of different types, and data in a variety of different formats.

The user profile/behavior data 402 may include data and insights about the user including name, unique user identifier, age, gender, race, interests, height, weight, risk score, profile photo, home address, work address, recently measured vital signs, etc. The user profile/behavioral data 402 may be provided by the user and stored in a database of all user profile data. The user profile/behavioral data 402 may also include behavioral preferences of the user. For example, communication preferences such as text, email, phone, in person; user preferences (e.g., phone call for upcoming reminders, etc.), appointment preferences (e.g., video call for virtual urgent care visits, in-person visits, telephone call visits, etc.), etc. The behavioral data 402 may also include implied or inferred preferences of the user for service delivery, responsiveness to notifications, responsiveness to suggestions, etc. Such behavioral data 402 may be implied and/or inferred based on other aspects of the user, such as age, gender, location, or other demographic data using one or more machine learning models, for example. Additionally, user profile/behavior data 402 may be imported from a third-party data source, in an embodiment. For example, one or more social media networking systems may capture user profile/behavior data 402 associated with the user. As another example, third-party systems, such as directories, fitness tracker databases, health activity tracking systems, and/or insurance systems may include user profile/behavior data 402 associated with the user.

The electronic health records 404 may include any medical data about the user. The data may be stored and accessed in a secure EMR/EHR data repository. The electronic health records 44 may include any medical information about particular user including diagnosed conditions (e.g., diabetic, mental health, heart attack, etc.), medical history, laboratory test results, treatment or care plans, and may be redundant or confirmatory of the data in the user profile 402. The electronic health records 404 may be part of other third-party servers 140 that are accessible with the user's permission via APIs 136.

The pharmacy data 408 may include any information about the medications that have been prescribed for the user. This could include historical prescriptions and current prescriptions. This may also include prescription related information such as drug interaction and refill dates. This may also include drug adherence information, whether the prescriptions are picked up in person and when, and any other information related to any prescriptions of the user.

The demographic data 410 may include general demographic information retrievable from a variety of governmental, public, or private data sources. The demographic information can be statistical data based on location, geography, race, gender, age, ancestry, etc.

The appointment data 412 may include any information or data regarding appointments. For example, the appointment data 412 may include number of missed appointments, the type of missed appointments, the time of missed appointments etc. The appointment data 412 may also include information about scheduled appointments. The information about scheduled appointments may include the time, date, and type of appointment as well as other information such as the service, the healthcare professional they will meet, etc. The appointment data 412 may also include information about available alternate appointments or appointments for other services that are available on the same day as their scheduled appointment.

The facility data 414 may include any information about the given facility. The information may include the services offered to patients, the IT capabilities of facility, the laboratory, pharmacy, and medical capabilities of a facility. For example, the facility data 414 may include the type of wireless communications for communicating with client devices 115, the locations of beacons or transceivers, other information about location determination, etc. The facility data 414 may change over time as different services and capabilities are possible by a given facility or removed from a given facility. In some implementations, the data storage 135 is coupled to receive the facility data from a server associated with each respective facility.

The clinical data 416 may include general clinical information about particular types of diseases or conditions, the best treatments for those diseases or conditions, preventive care, symptoms associated with diseases or conditions, etc. The clinical data 416 may also include preferred times for follow-up and delivery of particular services, as well as rankings of alternative treatments or services. For example, clinical data 416 may include a drug protocol regimen for treatment of a particular disease, such as hypertension. The drug protocol may include dosage amounts and timeframes for follow-up and delivery of additional diagnostic or other services. In an embodiment, a machine learning model may be trained to determine a suggested drug protocol regimen for a patient based on the medication adherence of the patient and medication data collected from the patient.

The geographic data 418 may include geographical data that can be used in comparison to the geographic location data for a client user device 115 of a patient. For example, the geographic data 418 may include geographic information about a facility, more specific geographic information about where particular services are offered or delivered within the facility, geographic information about landmarks or other points of interest that can be used to guide a patient to the facility, and any other geographic information or data that may be used in calculations in combination with or without the geographic location data of the client device 115. In some examples, a comparison of the geographic data 418 to geographic information received from the client device 115 can be used to determine a position of a patient, time til they reach a facility, directions to guide the patient to the facility or directions to a part of this facility that delivers services relevant to the patient's treatment as part of their optimized care.

The user client device data 420 may include information about the client device. For example, the user client device data 420 may include the electronic serial number of the client device 115, the carrier service utilized by the client device 115, the operating system of the client device 115, Bluetooth or other wireless communication capabilities of the client device 115, the applications installed on the client device 115, etc.

The other data 422 may be any other types of data not fitting into one the above categories that may be used by the conversational care optimization module 110 for providing recommendations to the user and optimizing the users visit to a given facility location. For example, synced wearable fitness devices, synced third-party mobile health applications, fitness goals (e.g., gain physical mobility, lose weight, etc.), activities (e.g., number of physical therapy sessions, data related to other health-related nonmedical services, etc. may be included as other data 422, in an embodiment. Other data 422 may include messages to and from primary care providers about medication reconciliation, including information on medication adherence, dosages being taken, and so forth.

FIG. 5 shows one implementation of the care optimization engine 302. As illustrated in FIG. 5, the care optimization engine 302 may include a reminder module 502, a patient notification module 504, an application set up module 506, a service ordering module 508, a check-in module 510, a payment module 512, a form-filling module 514, a care prioritizer 516, a caregiver/family notification module 518, a care gap module 520, a wait-time module 522, and a way finder module 524. These components 502, 504, 506, 508, 510, 512, 516, 518, 520, 522, and 524 are coupled for communication with each other and with the other components of the conversational care optimization module 110 as will be understood from the descriptions below.

The reminder module 502 may include software and/or logic for generating reminders about upcoming appointments or other medical related tasks pertinent to the patient. The reminder module 502 is coupled for communication and interaction with the appointment determination module 306 to determine any upcoming appointments. The reminder module 502 generates and sends reminder messages to the patient notification module 504. The reminder module 502 accesses data preference of the user stored in the user profile/behavioral data 402 to determine a patient's preferences for the timing and frequency of receiving reminders. The reminder module 502 is coupled to provide these reminders to the patient notification module 504.

The patient notification module 504 may include software and/or logic for generating notifications to send to the application on the client device 115 of the patient. The patient notification module 504 also receives input from the application on the client device 115 of the patient. The patient notification module 504 is able to send in a variety of notifications and messages to the client device 115 to notify the patient about information, remind the patient about him coming appointments, notify the patient of ways to optimize her visit, or any other communication between the conversational care optimization module 110 and the patient. The patient notification module 504 is coupled to the other modules of the care optimization engine 302 as well as the other components of the conversational care optimization module 110. The patient notification module 504 is coupled by the communication unit 241 and the network 105 to the client device 115.

The application set up module 506 may include software and/or logic for installing the conversational care optimization module 110 on the client device 115 of the patient. The application set up module 506 can also cause software to be downloaded to the client device 115, initialized and made operational. The application set up module 506 includes routines to set up the application including identification of the patient, identification of other data sources and information such as their home address, work address, primary medical facility, and other information about the account they have with a medical service provider.

The service ordering module 508 may include software and/or logic for cooperating and communicating with the services provided by medical services providers to order services for a particular patient. Part of optimizing a visit, maybe ordering services to be performed before or after an existing appointment. For example, the service ordering module 508 may order services such as additional clinical tasks, medical tasks, medical tests (X-rays, MRI, etc.), lab orders, refilling prescriptions, or other service needs when the patient is experiencing a hospital visit. The service ordering module 508 is coupled to send and receive messages to and from the client device 115 of the patient via the patient notification module 504.

The check-in module 510 may include software and/or logic for performing an automated check-in service. For example, the care optimization engine 302 may be notified that a patient is on site at a given facility. The check-in module 510 receives a signal that indicates that the patient is on-site at a facility and automatically performs any check-in procedures or processes needed to check a patient in for their next upcoming appointment at the facility. Once the check-in module 510 has completed the check-in process, the check-in module notifies the patient notification module 504 to send a message to the patient indicating that they have been checked in for their appointment. In some implementations, the check-in module 510 may cooperate with the other components of the care optimization engine 302 to perform any additional necessary steps to complete check-in. For example, if payment is required or additional paperwork or releases need to be signed, the check-in module 510 may cooperate with those other modules to make sure all steps necessary for check-in are completed.

The payment module 512 may include software and/or logic for collecting payment, charging insurance, or other payment activity that may need to be performed for a patient to complete an appointment. In order to make the patient visit seamless, the patient module 512 may automatically process any payment or co-pay, or any other payment required of the patient to complete the appointment. The payment module 512 may cooperate with the patient notification module 504 to generate notifications, acceptance, and confirmations of payments. The payment module 512 may be coupled to receive financial information from the user profile 402, e.g., the credit card to be charged for a co-pay for the current visit.

The form-filling module 514 may include software and/or logic for sending messages to and receiving information from the patient via their client device 115. Depending on the nature of the patient visit to the facility, additional information about their current condition, payment information, prior treatments or medical history, or other information may need to be completed for a particular form that is required for a particular appointment. In some implementations, the form-filling module 514 processes the information available for a patient and the information required for a given form. The form-filling module 514 may communicate with the patient via the client device 115 prior to their appointment to make sure all information necessary to complete required forms and paperwork has been received from the patient. In other implementations, the form-filling module 514 receives a signal that indicates when the patient arrives on site at a facility. In response to that signal, the form-filling module 514 confirms that all information necessary for the scheduled appointment are in the conversational care optimization module 110. If not, the form-filling module 514 interacts with the patient via their client device 115 to capture the missing information necessary for their appointment, so that they can immediately proceed to their appointment without having to wait or provide additional information. For example, the form-filling module 514 may completed forms needed to find appointments for the care gaps such as booking a COVID shot, booking a flu shot, or booking a medical test like a mammogram. In another example, the form-filling module 514 completes check-in forms for in-person appointments so the check-module 510 can generate a “Skip the line” pass and have the member go straight into the room for the appointment.

The care prioritizer 516 may include software and/or logic for determining which one or more tasks to recommend to a patient that they perform during their visit. The care prioritizer 516 is coupled to receive information from the care gap module 520 regarding tasks that the patient should be performing. In some implementations, the care prioritizer 516 is coupled to receive information from the patient needs module 308 to receive user input about what the patient believes are the most important additional tasks to be performed. The care prioritizer 516 orders and ranks the tasks based on a variety of considerations, including but not limited to, medical importance, facility availability, patient preference, time required in time available, etc. The care prioritizer 516 is coupled to provide one or more tasks for presentation to the patient using the patient notification module 504.

The caregiver/family notification module 518 may include software and/or logic for notifying other individuals that the patient has a designated. For example, a patient may input a preference one or more members of their family or a caregiver be notified in the same way that the conversational care optimization module 110 notifies the patient. The patient may indicate that all notifications or a subset of notifications are provided by the caregiver/family notification module 518 to the persons designated. The caregiver/family notification module 518 prepares and sends the notification messages to the persons designated. The caregiver/family notification module 518 is coupled to communicate outside of the care optimization engine 302 in a similar manner to the patient notification module 504 as is been described above.

The care gap module 520 may include software and/or logic for determining gaps in medical care for a given patient. The care gap module 520 accesses the medical information, appointment information, clinical data, user profile/behavioral data and other information stored in the data storage 135 and determines any particular tasks that may be performed to improve the health of the patient. The tasks may include any task such as getting an immunization, picking up prescriptions for better medical adherence, performing blood tests or other tests, etc. The care gap module 520 generates a list of recommended tasks that the patient performs to improve their health. This list of recommended tasks is provided by the care gap module 520 to the care prioritizer 516 so that notification messages can be generated and sent to the patient. In some implementations, the analysis in determinations made by the care gap module 520 and the care prioritizer 516 are performed using artificial intelligence or machine learning using machine leaning models.

The wait-time module 522 may include software and/or logic for determining the wait time for a given service. The wait-time module 522 is coupled to other systems of the medical facility to receive wait times for receiving different services. The wait-time module 522 is coupled to receive information about the patient's scheduled appointments, retrieve wait times and send those wait times to the patient notification module to be send to the patient. The wait-time module 522 can do this before the patient is due at a medical facility as well as once the patient is on site. The wait time information from the wait-time module 522 can also be provided to other components of the care optimization engine 302 so it can be used in their operations such as what care gaps can be done given wait times, and what care should be prioritized given wait times.

The way finder module 524 may include software and/or logic for providing additional information to the client device 115 to guide the patient to a particular destination. The way finder module 524 may be available offline, onsite, or both. The way finder module 524 may retrieve and provide maps with Points of Interest and/or indoor and outdoor navigation members movement to and from a destination of care, testing, or services. The way finder module 524 is coupled to retrieve this information from the data storage 135 and provide it to the patient via their client device 115.

In some implementations, the machine learning models for care prioritization and care gap analysis may be a neural network model and includes a layer and/or layers of memory units where memory units each have corresponding weights. A variety of neural network models may be utilized including feed forward neural networks, convolutional neural networks (CNN), recurrent neural networks, radial basis functions, other neural network models, as well as combinations of several neural networks. Additionally, the machine learning model may represent a variety of other machine learning techniques in addition to neural networks, for example, support vector machines, decision trees, Bayesian networks, random decision forests, k-nearest neighbors, linear regression, least squares, hidden Markov models, other machine learning techniques, and/or combinations of machine learning techniques. For example, the machine learning model may be a trained natural language understanding (NLU) model that is able to classify user input utterance (e.g., text or speech) and one or more of patient member profile, past medical and clinical history, user interaction history on different channels, demographic data, etc. to identify intent of messages (e.g., with a classification score) of a patient member, intent to schedule, reschedule or cancel an appointment, intent to refill a medical prescription, patient validation, status check, referral, information retrieval, COVID screening, location, leave a message for a primary care physician, etc. The one or more machine learning models may also support multi-lingual input classification. For example, the NLU models may be trained to be native language-specific and extended to support multi-lingual user inputs in English, Spanish, Vietnamese, Russian, etc. In another example, the machine learning models perform language translation of user inputs. In other examples, the NLU models may be trained based on the following features or attributes, including but not limited to: patient member profile (age, gender, location, race, socioeconomic, etc.); patient member clinical context (e.g., past and current health conditions, medications, allergies, laboratory results, treatments, historical encounter data, current encounter data, etc.); medical terms and dictionary; or statistics of input text, predicted disease conditions, proposed and actual dispositions. In some implementations, the NLU model is augmented or enhanced with a knowledge base that includes a medical dictionary, index search terms and associated dispositions for those terms. The knowledge base may also be enhanced to include data such as text input matches to text input frequency, disease frequency, drift between prediction and actual disposition, disease severity, patient member profile (e.g., age, gender, location, language, and other demographic data, etc.), physician or subject matter expert inputs. One or more machine learning models may be generated based on the user profile/behavior data 402, electronic health records 404, pharmacy data 408, demographic data 410, appointment data 412, facility data 414, clinical data 416, geographic data 418, user client device data 420, and/or other data 422.

In an embodiment, a care optimization engine 302 may use a machine learning model to determine one or more tasks that, if performed by the patient, would contribute to improving the health of the patient. For example, an annual wellness exam by the patient's medical provider may be a task that contributes greatly to improving the health of the patient because various tests and screenings for diseases may be performed as follow-up actions, post-visit. However, the patient may not schedule the various tests and screenings, post-visit. Instead, the patient may have a scheduled pick up of a prescription drug refill at a facility that has available appointments for the follow-up actions. In another embodiment, a care optimization engine 302 may use a specifically trained machine learning model to take, as input, medication data and care provider information that has been collected from a patient through a conversation interface application and, along with other data accessible by the care optimization engine 302, generate one or more care optimizations that include, as text output, specific recommended tasks for the patient to perform or other information related to the medication data and the care provider information. For example, a new medication may be available to the patient that is generic, providing cost savings to the patient. The new medication may be recommended to the care provider once the patient's medication has been reconciled, providing the prompt to the care provider automatically. As another example, other medications may be available for treating a specific diagnosis of the patient that can be provided in response to user input from the patient that indicates a request for alternative medications. As yet a further example, a specific machine learning model may determine that, when presented with the option to schedule follow-up tests and screenings, a high number of users schedule and complete the additional appointments. Other machine learning models may be generated to identify gaps in care for patients. As another example, a screening for prostate cancer may be recommended for men over a certain age based on other risk factors. The care optimization engine 302 may utilize one or more machine learning models to determine that patients with a particular set of demographic data, such as age, race, language capacity, and location, may respond better to a medical practitioner calling the patient to remind them to attend an appointment for the screening for prostate cancer in person at a facility. Thus, an action to schedule the appointment for the screening for prostate cancer may be delivered via a phone call from the medical practitioner in conjunction with a recently completed visit based on a machine learning model that ranks the screening as a priority based on the risk factors of the patient, in an embodiment.

As a further example, sentiment around the patient experience may be influenced by how regularly a physician or a primary care provider asks about medication reconciliation. A group of patients may be surveyed to answer one or more questions about sentiment around medication reconciliation. A machine learning model or other specialized model may be generated to identify one or more factors that may increase the sentiment of the surveyed patients, such as regular reminders before a visit to fill out a form or respond to prompts regarding medication adherence. The care optimization engine 302 may generate one or more machine learning models to increase and improve sentiment as well as medication adherence based on the automatic medication reconciliation methods, techniques, and processes described herein as provided by the automatic medication reconciliation module 112.

In some implementations, a machine learning model may be trained using any one of at least one of supervised learning (e.g., support vector machines, neural networks, logistic regression, linear regression, stacking, gradient boosting, etc.), unsupervised learning (e.g., clustering, neural networks, singular value decomposition, principal component analysis, etc.), or semi-supervised learning (e.g., generative models, transductive support vector machines, etc.). Additionally, or alternatively, a machine learning model may be trained using tensor networks. Different types of tensor networks, such as projected entangled-pair states (PEPS) and multi-scale entanglement renormalization ansatz (MERA), may be used to organize different types of information. Tensor networks may be used to capture weights of a neural network layer, allowing for efficient matrix multiplication during forward and backward propagation. Tensor networks may be used for various purposes, including data organization. For example, the care optimization engine 302 may mine the data storage or sources 135 to build links across the objects, such as patient users and identified gaps in care post-visit, based on features or attributes that they share with each other in order to train one or more machine learning models. As another example, one or more tensor networks may be used with a machine learning model to map out and approximate treatments, including drug protocol regimens, for various diseases or conditions, also given various demographics, geographic locations, clinical data, as well as other information available. By using tensor networks, data may be stored in multiple dimensions in a structured manner, enabling calculations and probability models to be generated across the multiple dimensions. Machine learning libraries like TENSORFLOW and PYTORCH provide functionalities to perform operations on tensors, like element-wise multiplication, matrix multiplication, and summation. These operations are fundamental to training neural networks used for machine learning. For example, users may be clustered based on having similar profile information, such as age, race, geographic location, and similar clinical data, such as shared diagnoses like obesity, high blood pressure, and high cholesterol. A machine learning model may be trained on the dataset of patients having similar profile information and similar clinical data and known outcomes based on medications prescribed as well as appointment behavior (e.g., likelihood of successfully completing an appointment) to determine which medications should be prescribed to this specific cluster of patients. This dataset of patients may be stored using one or more tensor networks where each dimension of the tensor network represents a dimension of information being stored, such as different types of profile information, clinical data, known outcomes of prescribed medications, appointment behavior, and so forth.

In some implementations, keyword-based database lookups, or search by keywords and clinical terms may be used to implement training of one or more machine learning models. For example, keyword extraction may be used for unsupervised training of classification of user input. User input, in the medication reconciliation context, may be classified into various clusters, such as requests for new medication, side effects of prescribed medications, non-adherence to medications, and other comments regarding medication that may include sentiment. In some implementations, one or more machine learning models may be trained to perform a single machine learning task or a variety of machine learning tasks. In other implementations, a machine learning model may be trained to perform multiple tasks. In yet other implementations, a machine learning model may be trained to receive the requested data and generate the response data. For example, a machine learning model that uses a neural network, or a machine learning neural network is a type of machine learning model that processes data and learns patterns, enabling computers to make complex decisions without explicit programming. Thus, given a requested data, such as a list of alternative medications for a diagnosis of attention deficit hyperactivity disorder, a machine learning model that uses a neural network that has been trained and receives different types of information about patients with that diagnosis and outcomes based on different prescribed medications can generate the list of alternative medications.

A plurality of training instances or samples from a training dataset may be determined from data storage or sources 135. A training instance may be applied as input to a machine learning model. Predicted machine learning model output may be generated by applying training input to a machine learning model. Additionally, or alternatively, the predicted machine learning model output may be compared with a known labelled output from the training instance and, using the comparison, one or more weights in the machine learning model may be updated. In some implementations, the one or more weights may be updated by backpropagating the difference over the entire machine learning model.

In some implementations, a trained machine learning model may be tested and updated, accordingly. A training dataset may be partitioned into a testing dataset and a training dataset. A testing instance may be applied from the training dataset as input to the trained machine learning model. A predicted output generated by applying a testing instance to the trained machine learning model may be compared with a known output for the testing instance to update an accuracy value (e.g., an accuracy percentage) for a machine learning model. In some implementations, the machine learning model may be versioned and serviced through an internal HTTP endpoint to be used by other component(s) of the conversational care optimization module 110. For example, once a model is trained and tested and determined to have acceptable accuracy (e.g., accuracy score satisfying a threshold), the model is pushed to the care optimization engine 302 for consumption. In some implementations, model development is an iterative process with retraining, testing and publishing steps performed iteratively, and adapted automatically to improve scores and accuracy. New versions will be published based on improvements and retraining using historical data and efficiency calculations as more data (e.g., feedback) is collected over a period of time. Feedback data may be used to develop new versions of models, in an embodiment. For example, the model or classifier class labels (e.g., an appointment for a service addressing a gap in care associated with a user to be recommended) requires clinical and business oversight and will be promoted for usage by capability based on clinical and business review. Continuous retraining using training data may be performed based on curation as part of clinical and data analysis.

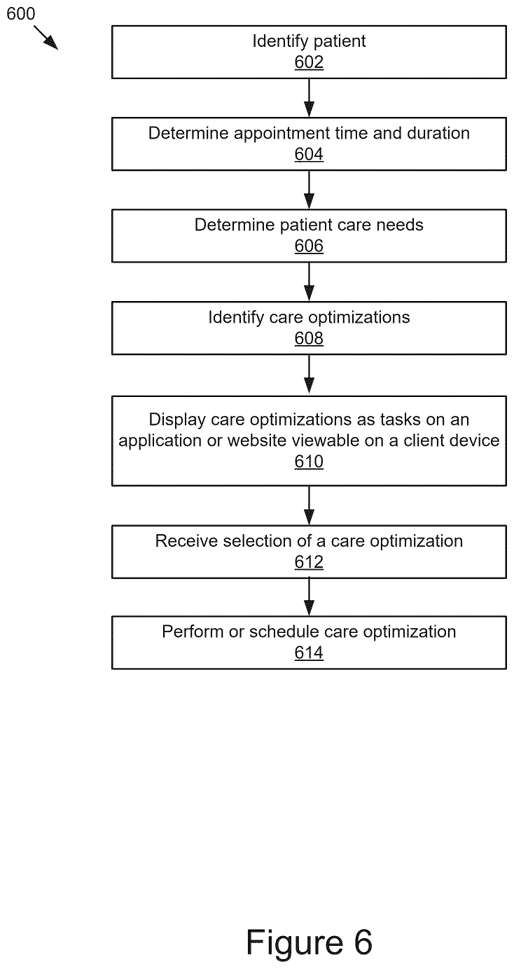

FIG. 6 is a flowchart illustrating one implementation of a general method 600 for optimizing post-visit actions. The method 600 begins by identifying 602 a patient. In some implementations, identifying information of the client device 115 is transmitted and can be used to look up an identify of the patient corresponding to the client device 115. In some implementations, an application installed on the client device 115 includes the identity of the patient associated with the client device 115. Once the patient associated with the client device 115 has been provided, it can be used in block 602 to identify the patient with specificity. Next, the method 600 determines 604 an appointment time and duration for the patient. The conversational care optimization module 110 may use the identity of the patient to determine the appointment for the patient by accessing a database including all appointments for the facility. Next, the method 600 determines 606 the care needs for the patient. For example, the method 600 can use the care optimization engine 302 to determine additional patient care needs. Next the method 600 identifies 608 possible care optimizations for the patient. The care optimizations may be based on the additional services in the care plan for the patient that are most urgent or prioritized. In some implementations, the care optimizations may be additional services, such as immunization services, that would avoid the patient needing to return to the facility for those services. Once one or more care optimizations have been identified, the method 600 displays 610 the one or more of those care optimizations as tasks on an application or website viewable by the user on their client device 115. For example, a notification of a new task may be sent to the user on their client device 115, such that upon opening a website or an application on their client device 115, the task is displayed indicating that a medication reconciliation process should be completed before a visit. The medication reconciliation process may be completed by responding to prompts through a conversational interface application, for example. The method 600 continues at step 612 with the conversational care optimization module 110 receiving a user selection of one of the care optimization choices presented in block 610. For example, the task may be completed by the user, indicating selection of the care optimization to perform the medication reconciliation process. Then the method 600 continues by performing or scheduling 614 the care optimization to be performed. In some implementations, the method 600 advantageously schedules the care optimization service to be performed immediately before or immediately after the service for which the patient is visiting the facility. Thus, the conversational care optimization module 110 advantageously avoids the patient having to return to the facility, and also eliminates any follow-up required for the additional care delivered during the visit.

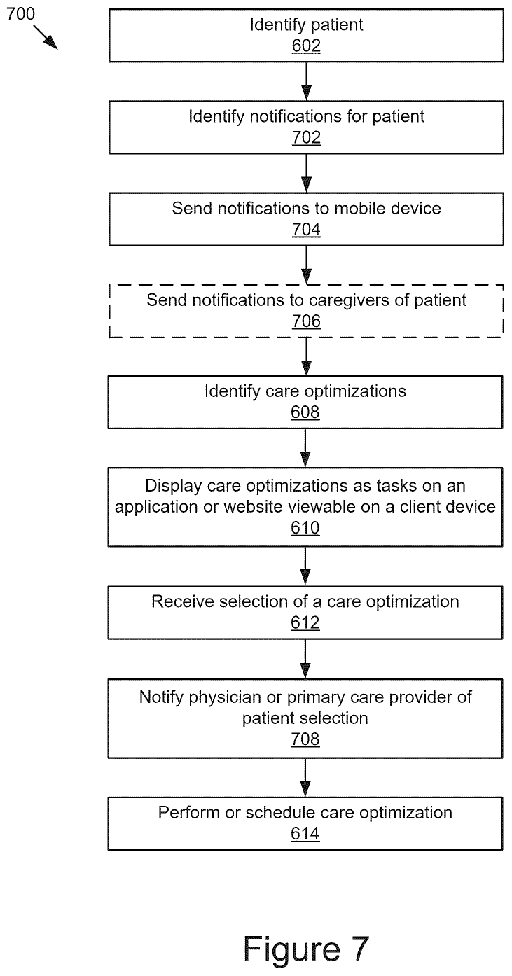

Referring now to FIG. 7, one implementation of a method 700 for notifying a patient and optimizing care is described. The method 700 begins similar to the method 600 described above with reference to FIG. 6 by identifying 602 the patient. The method 700 continues by identifying 702 notifications for the patient. Those notifications may include identification of additional services that could be provided, options to check-in for an existing appointment, confirmation the user has been checked in, information about their appointment such as a delay, etc. Next, the method 700 continues by sending 704 the notifications identified in block 702 to the client device 115 of the patient. Although not shown in FIG. 7, the patient may respond to the notifications presented on the client device 115. For example, if the notifications are informational only, they can be discarded or ignored. If the notifications require confirmation or input by the patient, the patient can input the confirmation or other information into the client device 115 and send it back to the conversational care optimization module 110. After block 704, the method 700 can optionally send 706 notifications to caregivers or family members of the patient. If the patient has set up this option, the conversational care optimization module 110 will send the same notifications to the family members or caregivers as are sent to the patient. In some implementations, the patient can filter or select which notifications are provided to their caregivers or family members. As shown in FIG. 7, this block is illustrated with dashed lines to indicate that this step is optional. After block 706 (or block 704), the method 700 continues similar to the method 600 described above with reference to FIG. 6 by performing the steps and blocks 608, 610, and 612 as has been described above. The method 700 described above with reference to FIG. 7 is particularly advantageous because the patient can be notified of information about their current appointment, and in real-time on the fly decide to optimize their visit by performing additional services in place of or in addition to an existing appointment. For example, in response to a notification that a process for medication reconciliation should be completed before the visit, a user may select to complete the medication reconciliation process through the conversation interface application and complete the task. Once that task is completed, the method 700 continues and, at step 708, notifies the physician or primary care provider of the patient selection and completion of the process for medication reconciliation. Then, at step 614, after step 708, the care optimization is performed or scheduled. In this example, the data is stored at the data storage 135 to update the medication list and adherence for the patient.

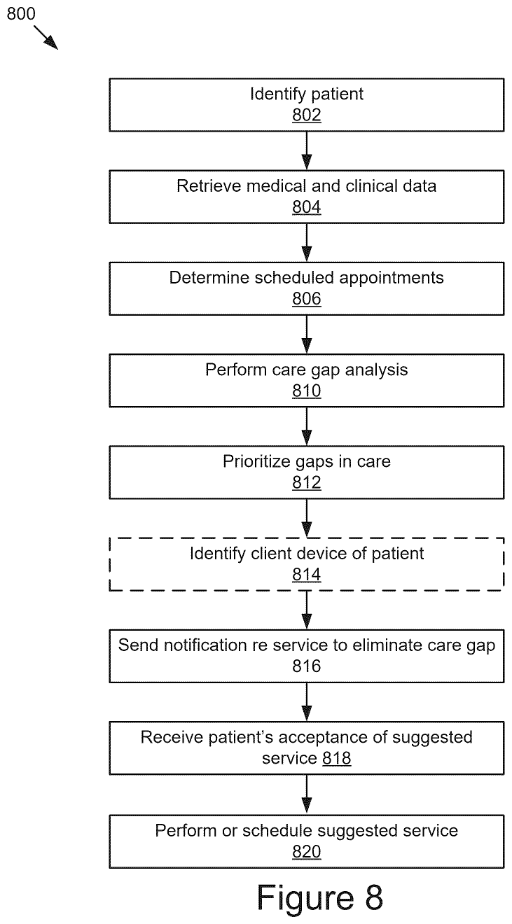

Referring now to FIG. 8, one implementation of a method 800 for determining care gaps and prompting a patient will be described. It should be understood that the method 800 of FIG. 8, may be performed with or in addition to the methods 600, 700 described above with reference to FIGS. 6 and 7, respectively. The method 800 begins by identifying 802 the patient. Next, the method 800 retrieves 804 medical and clinical data. The medical data may be data associated with the medical condition and treatment of the patient. The clinical data may be information about the particular condition and various treatment options for treatment of that condition. The method 800 continues by determining 806 the scheduled appointments for the patient. Next, the method 800 performs 810 care gap analysis. For example, the method 800 may determine instances where the patient has missed appointments, not performed recommended treatments, etc. In block 810, the method 800 identifies care gaps in the treatment of the patient. The method 800 next, prioritizes 812 the gaps in medical care from the most urgent that needs to be performed to the least urgent that needs to be performed. Next, the method 800 identifies 814 a client device 115 of the patient. In some implementations, where the patient has identified the client device 115 as her preferred communication channel, the client device 115 is used as the means to communicate with the patient. Since the geolocation and visit optimization module 110 may communicate with the patient through a variety of different means including telephone, mail, or other communication mechanisms rather than their mobile phone, block 814 is illustrated as optional in FIG. 8 with dashed lines. The method 800 continues by sending 816 a notification regarding a service, which if performed, will eliminate the care gap. In some implementations, the notification is the scheduling of an appointment or other delivery of the service. The method 800 continues by receiving 818 the patient's acceptance of the suggested service. Then the method 800 performs or schedules 820 the suggested service. This method 800 is particularly advantageous because the conversational care optimization module 110 can automatically identify gaps in the treatment of a patient and schedule them for delivery. This includes identifying that medication reconciliation has yet to be performed and including an option for the patient to complete that task before a visit or capturing data after a visit, such as messages back and forth to and from the primary care provider about the medication reconciliation. For example, if the patient gets put on new medication to treat a diagnosis and the new medication causes unwanted symptoms such as headache, nausea, and upset stomach, the patient may record those side effects through the medication reconciliation process, such as through a conversational user interface in which the patient user inputs text regarding the side effects to one or more medications. Additionally, through the medication reconciliation process, the patient may identify new medications that they would like to try to tackle issues pertaining to their health that may or may not have received a diagnosis, such as mental health issues, weight loss, and pre-diabetes. For example, through the same conversational user interface, the patient may input text that indicates that they want to try a new medication for their attention deficit hyperactivity disorder diagnosis because the medication they are using currently has an unwanted side effect of raising their blood pressure. Previously, the patient would need to set up a medical appointment to bring up this request. However, in an embodiment, the patient may, through the conversational user interface, make this request and supply other information, such as a photo of their current blood pressure readings from a blood pressure monitor. The conversational user interface, using one or more machine learning models, may automatically book a new appointment with a medical provider based on this request, attaching the photo of the blood pressure reading and the user input regarding the request for new medication. This beneficially enables primary care providers to be prepared to address the reasons why certain medications may or may not be advisable for a patient.

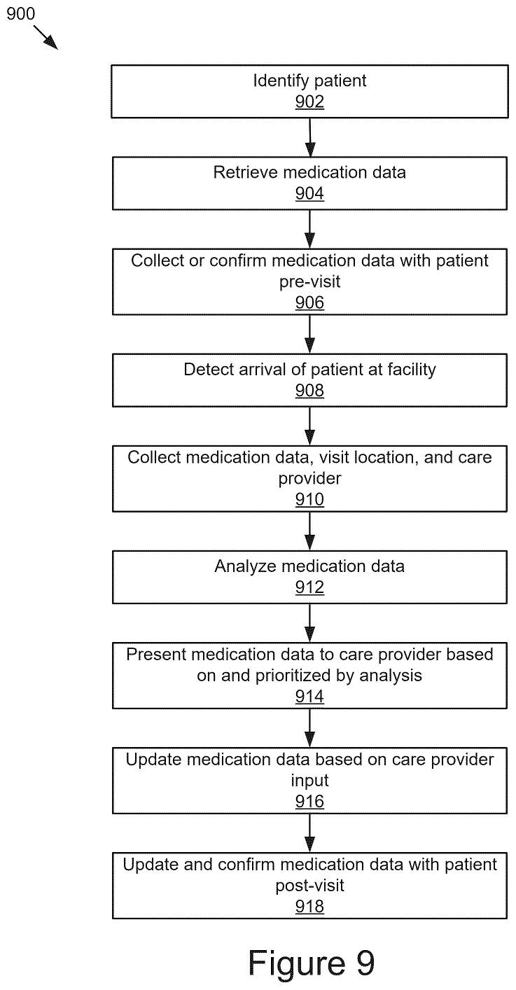

Referring now to FIG. 9, one implementation of a method 900 for reconciling medication with a patient automatically will be described. It should be understood that the method 900 of FIG. 9, may be performed with or in addition to the methods 600, 700, 800 described above with reference to FIGS. 6, 7 and 8, respectively. The method 900 begins by identifying 902 the patient. Next, the method 900 retrieves 904 medication data. The medication data may be data associated with the medications that have been prescribed to the patient as well as self-reported medications, drugs, vitamins, and supplements. The medication data may include related clinical data about the particular condition and various treatment options for treatment of that condition. The method 900 continues by collecting or confirming 906 medication data with the patient pre-visit. For example, this includes prompting the patient through a notification on a mobile phone to fill out a medication reconciliation form or engaging in a conversation interface application regarding the medication data. Additionally, as described above, the conversation interface application may include additional prompts, such as “Are you experiencing any side effects with this medication?” and “Is there anything else you′d like to bring up regarding your medications?” Based on the responses to such prompts, the conversation interface application may update the patient's records, accordingly. Other actions may automatically happen based on the responses, such as generating a prompt to the medical provider to ask a specific question regarding a medication, creating a new appointment to refill a prescription, generating a reminder to set up a new medical appointment in three months for a check-up, automatically generating a new task for the user to perform, automatically generating a reminder for the user, and so forth. As a result, the implementation results in an improved user experience and the conversational interface application increases the number of care optimizations made available to the user as well as reducing the amount of time to deliver the care optimizations, in an embodiment.

Next, the method 900 detects 908 arrival of patient at facility. For example, a third party system or another module connected to the healthcare management server 120 through network 105 may provide a signal indicating that the patient has arrived at a particular facility. In an embodiment, the method 900 may determine the arrival of the patient at a facility using location-based technology, such as GPS. In block 910, the method 900 collects medication data, visit location and care provider information. This data may be collected from one or more data sources, such as the healthcare management server 120, data storage or sources 135, third party servers 140, and/or client devices 115. For example, the medication data collected at block 910 may include a history of medications that have been prescribed to the patient. As another example, the visit location and care provider information collected at block 910 may further include a geographic location of where the visit took place and the credentials of the care provider, such as their title, educational experience, and work experience. In an embodiment, one or more artificial intelligence/machine learning (AI/ML) models may use the collected medication data, visit location data, and care provider information to provide one or more suggestions to the care provider via a medication reconciliation interface on a client device associated with the care provider assigned to the patient. The method 900 next analyzes 912 the medication data. In an embodiment, a knowledge base of medications, ontology, and other data from various sources may be used to analyze the medication data in step 912. Additionally, for example, this may include identifying any updates in medications being taken, including dosage information and medication adherence, as well as requests for discussion by the patient and new medications being requested. Next, the method 900 presents 914 medication data to care provider based on and prioritized by analysis. For example, if a new medication is requested, additional analysis may be performed by one or more AI/ML models, in an embodiment, to identify any possible side effects from combinations of medications and/or conditions and provide one or more suggestions to the care provider based on the analysis. As another example, an update by a patient saying that they have stopped taking a prescribed medication may be highly prioritized based on the condition of the patient. For example, if the care provider had prescribed a medication to prevent the onset of a chronic disease, such as diabetes, and the patient indicates through the automated medication reconciliation process that they have stopped taking the medication due to cost, the care provider may try to identify cheaper alternatives to the prescribed medication or identify other ways to prevent the onset of diabetes. The method 900 continues by updating 916 medication data based on care provider input. In the care provider's user interface, changes and notes may be made during the clinical visit, such as deleting a medication or altering the dosage of a medication. These changes and notes may be stored as new updated medication data in association with the patient member's user profile in the data storage 135. The method 900 continues by updating 918 and confirming the medication data with the patient post-visit. This may be executed through another notification or alert to the patient through their client device 115 to update or confirm 918 the medication data.

Referring now to FIGS. 10A-10B and 11A-11C, example implementations of graphic user interfaces that may be produced by the conversational care optimization module 110 and, in particular, the user interface engine 308, will now be described. While the user interfaces will now be described primarily in the context of providing additional services such as medication reconciliation, it should be understood that the methods of the present disclosure may be applied similarly to send notifications about any medical condition and prompt the appointments or service treatments applicable to any such medical conditions. FIGS. 10A-10B illustrate example user interfaces shown on a client device 115 such as a smart phone.

FIGS. 10A-10B illustrate example implementations of graphical user interfaces 1000 and 1050 of an application on a client device 115 that implements the processes of the conversational care optimization module 110 and the automatic medication reconciliation module 112. As displayed on FIG. 10A, the home screen of the graphical user interface 1000 shows a notification in a dialog 1002. The dialog 1002 may be selected by the user on the client device 115 that opens the conversational interface application. The second screen of the example graphical user interface 1000 includes a “Hi, Neil” welcome message and enables the user to select a reminder 1004 that the user has an appointment today and an icon to “View Appointments” may be selected. This takes the user to the third screen illustrated in FIG. 10A where the viewing user may see a section header 1006 that states “Outstanding Pre-Visit Tasks” that are suggested to be completed before the appointment. A link 1008 to a form to “Review Medication List” may be selected by the viewing user, which, upon selection, presents example user interface 1050 shown in FIG. 10B that includes a list of prescribed medications and patient reported medications.

As shown in FIG. 10B, example user interface 1050 includes a section 1052 for a prescribed medication, “Hydrochlorothiazide 25 Mg Tablet” as well as a photo 1058 of the tablets. A prompt 1054 with radio button response options asks the viewing user “Are you taking Hydrochlorothiazide?” with the options being either “Taking” or “Not taking.” Additionally, optional check boxes 1056 enable the user to select whether they want to discuss the medication with the care team and/or request a refill of the medication. A patient reported medication is shown in a new section 1060, “Aspirin 100 Mg tablet” along with a photo 1066 of the tablets. Similarly, a prompt 1062 asks whether the patient is taking Aspirin with a radio button response of “Taking” or “Not taking” and a checkbox 1064 that optionally requests to discuss the medication with the care provider. An optional checkbox 1068 is also presented in the user interface 1050 that reads “Remove this medication from my list.” A button 1070 may be selected to continue to the review screen, the second screen shown in FIG. 10B. In that screen, a button 1072 may be selected to “Submit medication list” or another button 1074 may be selected to “Go back and change answers.” In this way, the viewing user may, through this conversational interface application, go through each medication on their own time before a visit to reconcile which medications they are currently taking. Once the medication list is submitted, after selecting button 1072, confirmation screen 1076 is shown, indicating that the “updates have been saved and will be reviewed by your care team.”

Referring now to FIGS. 11A-11C, example implementations of graphical user interfaces 1100, 1130 and 1160 of an application on a client device 115 that implements the processes of the automatic medication reconciliation module 112. As displayed on FIG. 11A, the home screen of the graphical user interface 1100 shows a notification banner 1102 that states “External medications need attention.” Additionally, a button 1106 labelled “Go Reconcile” is included within the notification banner 1102. A listing of medication data associated with the patient is shown in table 1104. Though not shown, other information may be retrieved from data storage or sources 135 and displayed for presentation to the care provider in the graphical user interface 1100. For example, as described above, one or more suggestions may be provided for display to the care provider based on one or more user inputs received from the conversation interface application. In an embodiment, the one or more suggestions may be provided to inform the care provider of a request for a new medication, a list of alternative medications that may be selected by the care provider based on the request for new medication, a list of possible side effects based on the medications being taken by the patient, and a list of non-medication related behaviors and/or therapies that may be suggested to the patient based on the symptoms reported in relation to the medications being taken.

FIG. 11B illustrates a portion of graphical user interface 1100 as well as a new dialog screen 1130. In this dialog screen 1130, the care provider may create a new note or review past notes. As shown in FIG. 11B, a listing of medications 1132 is shown as well as comments 1134 from the patient as gathered from the conversational interface application described above and with respect to FIGS. 10A-10B. For example, medications A, B, and C are notated with “Taking” while medication D is notated with “Wants to discuss.” Medication E includes a comment of “Not taking” and an “X” may appear for the care provider to consider removing medication E from the list of medications that the patient is actively taking. Medication F may be a new medication that the patient has requested to treat a particular condition. Other user interface elements may be presented, though not shown in FIG. 11B, such as a plus “+” sign to add a new medication, a checkmark to indicate that a medication has been discussed, and text fields for the care provider to input additional information during the visit. For example, upon selecting a plus “+” sign to add a new medication, a list of suggested medications based on the medications being taken by the patient may be provided in a drop-down menu. The suggested medications may be generated by one or more AI/ML models based on the medication data and clinical data accessible about the patient, in an embodiment. In another embodiment, the care provider may be prompted with a graphical user interface dialog box if a medication was not discussed, as indicated by the checkmark being selected.

FIG. 11C illustrates the updated graphical user interface 1100 as user interface 1160. In the listing 1162 of medications, as a result of the care provider selecting the “X” to remove Medication E, indicates the action taken as a strikethrough of the medication. In other embodiments, the row for Medication E may simply be removed from the table 1164. In the table 1168, the list of medications is also updated. A notification banner 1166 may be included in graphical user interface 1160 to indicate that an update is needed because the dispense history has not been updated recently. Thus, medication reconciliation may be automatically and efficiently updated, improving the experience of the patient and improving medication adherence.

It should be understood that the user interfaces presented and discussed above with reference to FIGS. 10A-10B and 11A-11C are illustrative and provided merely by way of example. The user interfaces may be modified to provide additional or fewer buttons, additional or less content, and be related to other patient medications, conditions, symptoms and care optimization services beyond medication reconciliation. Moreover, the flow between the user interfaces described above with reference to FIGS. 10A-11C may be modified to include additional or fewer user interfaces.

A system and method for improved medical care powered by conversational care optimization for determining and providing the appropriate healthcare services has been described. In the above description, for purposes of explanation, numerous specific details are set forth in order to provide a thorough understanding of the techniques introduced above. It will be apparent, however, to one skilled in the art that the techniques can be practiced without these specific details. In other instances, structures and devices are shown in block diagram form in order to avoid obscuring the description and for ease of understanding. For example, the techniques are described in one implementation above primarily with reference to software and particular hardware. However, the present invention applies to any type of computing system that can receive data and commands, and present information as part of any peripheral devices providing services.

Reference in the specification to “one implementation” or “an implementation” means that a particular feature, structure, or characteristic described in connection with the implementation is included in at least one implementation. The appearances of the phrase “in one implementation” in various places in the specification are not necessarily all referring to the same implementation.

Some portions of the detailed descriptions described above are presented in terms of algorithms and symbolic representations of operations on data bits within a computer memory. These algorithmic descriptions and representations are, in some circumstances, used by those skilled in the data processing arts to convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing”, “computing”, “calculating”, “determining”, “displaying”, or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The techniques also relate to an apparatus for performing the operations herein. This apparatus may be specially constructed for the required purposes, or it may comprise a general-purpose computer selectively activated or reconfigured by a computer program stored in the computer. Such a computer program may be stored in a non-transitory computer readable storage medium, such as, but is not limited to, any type of disk including floppy disks, optical disks, CD-ROMs, and magnetic disks, read-only memories (ROMs), random access memories (RAMs), EPROMs, EEPROMs, magnetic or optical cards, flash memories including USB keys with non-volatile memory or any type of media suitable for storing electronic instructions, each coupled to a computer system bus.

The technology described herein can take the form of a hardware implementation, a software implementation, or implementations containing both hardware and software elements. For instance, the technology may be implemented in software, which includes but is not limited to firmware, resident software, microcode, etc. Furthermore, the technology can take the form of a computer program product accessible from a computer-usable or computer-readable medium providing program code for use by or in connection with a computer or any instruction execution system. For the purposes of this description, a computer-usable or computer readable medium can be any non-transitory storage apparatus that can contain, store, communicate, propagate, or transport the program for use by or in connection with the instruction execution system, apparatus, or device.

A data processing system suitable for storing and/or executing program code may include at least one processor coupled directly or indirectly to memory elements through a system bus. The memory elements can include local memory employed during actual execution of the program code, bulk storage, and cache memories that provide temporary storage of at least some program code in order to reduce the number of times code must be retrieved from bulk storage during execution. Input/output or I/O devices (including but not limited to keyboards, displays, pointing devices, etc.) can be coupled to the system either directly or through intervening I/O controllers.

Network adapters may also be coupled to the system to enable the data processing system to become coupled to other data processing systems, storage devices, remote printers, etc., through intervening private and/or public networks. Wireless (e.g., Wi-Fi™) transceivers, Ethernet adapters, and modems, are just a few examples of network adapters. The private and public networks may have any number of configurations and/or topologies. Data may be transmitted between these devices via the networks using a variety of different communication protocols including, for example, various Internet layer, transport layer, or application layer protocols. For example, data may be transmitted via the networks using transmission control protocol/Internet protocol (TCP/IP), user datagram protocol (UDP), transmission control protocol (TCP), hypertext transfer protocol (HTTP), secure hypertext transfer protocol (HTTPS), dynamic adaptive streaming over HTTP (DASH), real-time streaming protocol (RTSP), real-time transport protocol (RTP) and the real-time transport control protocol (RTCP), voice over Internet protocol (VOIP), file transfer protocol (FTP), WebSocket (WS), wireless access protocol (WAP), various messaging protocols (SMS, MMS, XMS, IMAP, SMTP, POP, WebDAV, etc.), or other known protocols.

Finally, the structure, algorithms, and/or interfaces presented herein are not inherently related to any particular computer or other apparatus. Various general-purpose systems may be used with programs in accordance with the teachings herein, or it may prove convenient to construct more specialized apparatus to perform the required method blocks. The required structure for a variety of these systems will appear from the description above. In addition, the specification is not described with reference to any particular programming language. It will be appreciated that a variety of programming languages may be used to implement the teachings of the specification as described herein.

The foregoing description has been presented for the purposes of illustration and description. It is not intended to be exhaustive or to limit the specification to the precise form disclosed. Many modifications and variations are possible in light of the above teaching. It is intended that the scope of the disclosure be limited not by this detailed description, but rather by the claims of this application. As will be understood by those familiar with the art, the specification may be embodied in other specific forms without departing from the spirit or essential characteristics thereof. Likewise, the particular naming and division of the modules, routines, features, attributes, methodologies and other aspects are not mandatory or significant, and the mechanisms that implement the specification or its features may have different names, divisions and/or formats.

Furthermore, the modules, routines, features, attributes, methodologies, engines, and other aspects of the disclosure can be implemented as software, hardware, firmware, or any combination of the foregoing. Also, wherever an element, an example of which is a module, of the specification is implemented as software, the element can be implemented as a standalone program, as part of a larger program, as a plurality of separate programs, as a statically or dynamically linked library, as a kernel loadable module, as a device driver, and/or in every and any other way known now or in the future. Additionally, the disclosure is in no way limited to implementation in any specific programming language, or for any specific operating system or environment. Accordingly, the disclosure is intended to be illustrative, but not limiting, of the scope of the subject matter set forth in the following claims.

What is claimed is:

1. A method comprising:
retrieving a plurality of data attributes associated with a visit for a patient;
collecting medication data and care provider information associated with the patient;
using one or more machine learning models, synthesizing one or more care optimizations based on the collected medication data and the care provider information as input to the one or more machine learning models;
generating text output at a user interface that includes the synthesized one or more care optimizations based on the collected medication data and the care provider information;
presenting the user interface including the generated text output for display on a client device associated with the patient;
receiving a selection of a care optimization from the client device; and
causing the care optimization to be performed.

2. The method of claim 1, further comprising:
determining clinical data associated with the patient, the clinical data including one or more diseases associated with the patient, one or more treatments for the one or more diseases, one or more symptoms associated with the one or more diseases and one or more drug protocol regimens for treatment of the one or more diseases associated with the patient;
selecting a machine learning model of the one or more machine learning models based on the clinical data associated with the patient;
determining a portion of the generated text output associated with the one or more care optimizations based on the selected machine learning model; and
presenting the user interface including the determined portion of the generated text output associated with the one or more care optimizations for display on the client device associated with the patient.

3. The method of claim 1, further comprising:
generating a notification regarding a suggested action based on priority;
sending the notification to the client device associated with the patient; and
receiving acceptance of the suggested action from the client device.

4. The method of claim 1, further comprising:
detecting arrival of the patient at a facility;
generating a notification at a client device associated with a care provider assigned to the patient at the facility, the notification including the medication data;
responsive to selection of the notification, presenting a medication reconciliation interface for display on a client device associated with the care provider assigned to the patient at the facility, the medication reconciliation interface including the selected care optimization by the patient.

5. The method of claim 1, further comprising:
analyzing the collected medication data;
generating additional text output associated with at least one of the one or more care optimizations associated with the collected medication data based on the analyzing; and
presenting the additional text output associated with the at least one of the one or more care optimizations in the user interface.

6. The method of claim 5, wherein the one or more care optimizations are prioritized by the analyzed medication data.

7. The method of claim 1, wherein the care optimization to be performed is prompting a care provider assigned to the patient to inform the patient of one or more alternative medications during the visit.

8. A system comprising one or more processors and memory operably coupled with the one or more processors, wherein the memory stores instructions that, in response to the execution of the instructions by one or more processors, cause the one or more processors to perform operations including:
retrieving a plurality of data attributes associated with a visit for a patient;
collecting medication data associated with the patient;
using one or more machine learning models, synthesizing one or more care optimizations based on the collected medication data and the care provider information as input to the one or more machine learning models;
generating text output at a user interface that includes the synthesized one or more care optimizations based on the collected medication data and the care provider information;
presenting the user interface including the generated text output for display on a client device associated with the patient;
receiving a selection of a care optimization from the client device; and
causing the care optimization to be performed.

9. The system of claim 8, wherein the operations further comprise:
determining clinical data associated with the patient, the clinical data including one or more diseases associated with the patient, one or more treatments for the one or more diseases, one or more symptoms associated with the one or more diseases and one or more drug protocol regimens for treatment of the one or more diseases associated with the patient;
selecting a machine learning model of the one or more machine learning models based on the clinical data;
determining a portion of the generated text output associated with the one or more care optimizations based on the selected machine learning model; and
presenting the user interface including the determined portion of the generated text output associated with the one or more care optimizations for display on the client device associated with the patient.

10. The system of claim 8, wherein the operations further comprise:
generating a notification regarding a suggested action based on priority;
sending the notification to the client device associated with the patient; and
receiving acceptance of the suggested action from the client device.

11. The system of claim 8, wherein the operations further comprise:
detecting arrival of the patient at a facility;
generating a notification at a client device associated with a care provider assigned to the patient at the facility, the notification including the medication data;
responsive to selection of the notification, presenting a medication reconciliation interface for display on a client device associated with the care provider assigned to the patient at the facility, the medication reconciliation interface including the selected care optimization by the patient.

12. The system of claim 8, wherein the operations further comprise:
analyzing the collected medication data;
generating additional text output associated with at least one of the one or more care optimizations associated with the collected medication data based on the analyzing; and
presenting the additional text output associated with the at least one of the one or more care optimizations in the user interface.

13. The system of claim 12, wherein the one or more care optimizations are prioritized by the analyzed medication data.

14. The system of claim 12, wherein the care optimization to be performed is prompting a care provider assigned to the patient to inform the patient of one or more alternative medications during the visit.

15. A method comprising:
generating a plurality of data attributes associated with a patient;
receiving medication data associated with the patient;
identifying a care provider at a facility based on an indication of arrival of the patient at the facility;
generating a user interface at a client device associated with the care provider that includes one or more care optimizations based on the received medication data;
presenting the user interface for display on the client device associated with the care provider;
receiving user input associated with a care optimization from the client device; and
causing the care optimization to be performed.

16. The method of claim 15, further comprising:
using one or more machine learning models, generating the one or more care optimizations based on an analysis of the collected medication data; and
prioritizing the one or more care optimizations based on an improvement in one or more patient outcomes.

17. The method of claim 15, wherein the care optimization to be performed is confirming the medication data with the patient after the visit.

18. The method of claim 15, wherein the care optimization to be performed is updating the medication data with the patient during the visit.

19. The method of claim 15, wherein the care optimization to be performed is generating a new machine learning model based on the received medication data.

20. The method of claim 15, wherein the care optimization to be performed is generating a determination of sentiment of user feedback received.
